(19) United States
(12) Patent Application Publication
(10) Pub. No.: US 2024/0101587 A1
(43) Pub. Date: Mar. 28, 2024

(54) BIOMASS REMOVAL BY CENTRIFUGATION

(71) Applicant: Glycom A/S, Hørsholm (DK)

(72) Inventors: Nikolay KHANZHIN, Humlebaek (DK); Marco WIESER, Village-Neut (FR); Jesper THØGERSEN, Varde (DK)

(21) Appl. No.: 18/257,986

(22) PCT Filed: Dec. 17, 2021
(86) PCT No.: PCT/IB2021/061934

§ 371 Date: Jun. 16, 2023

(30) Foreign Application Priority Data
Dec. 18, 2020 (DK) PA 2020 01431

(51) Int. Cl. C07H 1/06; C07H 3/06

(52) U.S. Cl. CPC C07H 1/06; C07H 3/06

(57) ABSTRACT

Disclosed herein are systems and methods for the purification of human milk oligosaccharides (HMOs). The HMOs can be purified from a suspension containing one or more of a bacterial biomass, cell fragments, small particles, and biomolecules. Advantageously, the solution can be pre-treated prior to centrifuging to form a clarified supernatant containing purified HMOs.

### FIELD

The disclosure relates generally to the removal of biomass via centrifugation of a pre-treated suspension.

### BACKGROUND

During the past decades, the interest in the preparation and commercialisation of human milk oligosaccharides (HMOs) has been increasing steadily. The importance of HMOs is directly linked to their unique biological activities, therefore HMOs have become important potential products for nutrition and therapeutic uses. As a result, low cost ways of producing industrially HMOs have been sought.

To date, the structures of more than 140 HMOs have been determined, and considerably more are probably present in human milk (Urashima et al.: Milk oligosaccharides, Nova Biomedical Books, 2011; Chen Adv. Carbohydr. Chem. Biochem. 72, 113 (2015)). The HMOs comprise a lactose (Galβ1-4Glc) moiety at the reducing end and may be elongated with an N-acetylglucosamine, or one or more N-acetyllactosamine moiety/moieties (Galβ1-4GlcNAc) and/or a lacto-N-biose moiety (Galβ1-3GIcNAc). Lactose and the N-acetyllactosaminylated or lacto-N-biosylated lactose derivatives may further be substituted with one or more fucose and/or sialic acid residue(s), or lactose may be substituted with an additional galactose, to give HMOs known so far.

Direct fermentative production of HMOs, especially of those being a trisaccharide, has recently become practical (Han et al. Biotechnol. Adv. 30, 1268 (2012) and references cited therein). Such fermentation technology has used a recombinant E. coli system wherein one or more types of glycosyl transferases originating from viruses or bacteria have been co-expressed to glycosylate exogenously added lactose, which has been internalized by the LacY permease of the E. coli. However, the use of a recombinant glycosyl transferase, especially series of recombinant glycosyl transferases to produce oligosaccharides of four or more monosaccharide units, has always led to by-product formation hence resulting in a complex mixture of oligosaccharides in the fermentation broth. Further, a fermentation broth inevitably contains a wide range of non-oligosaccharide substances such as cells, cell fragments, proteins, protein fragments, DNA, DNA fragments, endotoxins, caramelized by-products, minerals, salts or other charged molecules and numerous metabolites.

For separating HMOs from carbohydrate by-products and other contaminating components, active charcoal treatment combined with gel filtration chromatography has been proposed as a method of choice (WO 01/04341, EP-A-2479263, Dumon et al. Glycoconj. J. 18, 465 (2001), Priem et al. Glycobiology 12, 235 (2002), Drouillard et al. Angew. Chem. Int. Ed. 45, 1778 (2006), Gebus et al. Carbohydr. Res. 361, 83 (2012), Baumgärtner et al. ChemBioChem 15, 1896 (2014)). Although gel filtration chromatography is a convenient lab scale method, it cannot be efficiently scaled up for industrial production.

Recently, EP-A-2896628 has described a process for purification of 2′-FL from a fermentation broth obtained by microbial fermentation comprising the following steps: ultrafiltration, strong cation exchange resin chromatography (H⁺-form), neutralization, strong anion exchange resin chromatography (acetate-form), neutralization, active charcoal treatment, electrodialysis, second strong cation exchange resin chromatography (H⁺- or Na⁺-form), second strong anion exchange resin chromatography (Cl⁻-form), second active charcoal treatment, optional second electrodialysis and sterile filtration.

WO 2017/182965 and WO 2017/221208 have disclosed a process for purification of LNT or LNnT from fermentation broth comprising ultrafiltration, nanofiltration, active charcoal treatment and treatment with strong cation exchange resin (H⁺-form) followed by weak anion exchange resin (base form).

WO 2015/188834 and WO 2016/095924 have disclosed the crystallization of 2′-FL from a purified fermentation broth, the purification comprising ultrafiltration, nanofiltration, active charcoal treatment and treatment with strong cation exchange resin (H⁺-form) followed by weak anion exchange resin (base form).

Other prior art documents have disclosed purification methods elaborated for low lactose or no-lactose fermentation broths. According to these procedures, lactose added in excess during the fermentative production of a neutral HMO has been hydrolysed in situ after completion of the fermentation by the action of a β-galactosidase, resulting a broth that substantially does not contain residual lactose. Accordingly, WO 2012/112777 has disclosed a series of step to purify 2′-FL comprising centrifugation, capturing the oligosaccharide on charcoal followed by elution and flash chromatography on ion exchange media. WO 2015/106943 has disclosed purification of 2′-FL comprising ultrafiltration, strong cation exchange resin chromatography (H⁺-form), neutralization, strong anion exchange resin chromatography (Cl⁻-form), neutralization, nanofiltration/diafiltration, active charcoal treatment, electrodialysis, optional second strong cation exchange resin chromatography (Na⁺-form), second strong anion exchange resin chromatography (Cl⁻-form), second active charcoal treatment, optional second electrodialysis and sterile filtration. WO 2019/063757 has disclosed a process for purification of a neutral HMO comprising separating biomass from fermentation broth and treatment with a cation exchange material, an anion exchange material and a cation exchange adsorbent resin.

However, alternative procedures for isolating and purifying a neutral HMO from non-carbohydrate components of the fermentation broth in which they have been produced, especially those suitable for industrial scale, are needed to improve the recovery yield of the HMO and/or to simplify prior art methods while the purity of the HMO is at least maintained, preferably improved.

### SUMMARY

In its broadest aspect, the present invention relates to a method of purification of oligosaccharides from a suspension containing one or more of biomass and proteins, the method comprising:
pre-treating the suspension via pH-adjustment, dilution and/or heat treatment; and
centrifuging the suspension after the pre-treatment,
   thereby forming a clarified supernatant containing the purified oligosaccharides.

The suspension is preferably a fermentation broth.

The pre-treatment is preferably a pH-adjustment of the suspension, preferably a fermentation broth.

More preferably, the pre-treatment is pH-adjustment of the suspension with dilution and/or heat treatment.

Even more preferably, the pre-treatment of the suspension is pH-adjustment, dilution and heat treatment.

The oligosaccharides are preferably human milk oligosaccharides (HMOs).

The HMOs are preferably neutral HMOs.

The neutral HMOs are preferably selected from the group consisting of 2′-FL, 3-FL, DFL, LNT, LNnT, LNFP-I, LNFP-II, LNFP-III, LNFP-V and LNFP-VI.

### BRIEF DESCRIPTION OF THE FIGURES

FIG. 1 illustrates a graph of centrifugation time compared to supernatant clarity according to an embodiments disclosed in Example 5.

FIG. 2 illustrates a graph of settling velocities at different pH values according to Example 7.

FIG. 3 illustrates a flowchart according to one or more embodiments of the disclosure.

### DETAILED DESCRIPTION

Disclosed herein are embodiments of methods for removing biomass(es) from a heterogeneous solid-liquid mixture or system (suspension) containing dissolved organic molecules. The term “biomass” refers to non-dissolved solid particles in the liquid phase of the heterogeneous system and in the context of a fermentation, it refers to the suspended, precipitated or insoluble materials originating from fermentation cells, like intact cells, disrupted cells, cell fragments, proteins, protein fragments, polysaccharides; and in the context of an enzymatic reaction, it refers to (mainly denatured and/or precipitated) proteins or protein fragments originating from the enzyme used therein. Typically, the dissolved organic molecule(s) in a solid-liquid mixture or system is/are metabolic product(s) of a fermentation process wherein it/they is/are secreted into the fermentation broth or product(s) of an in vitro enzymatic reaction. For example, the biomass can be a biomass originated from bacterial or fungal cells and the solid-liquid mixture or system can be a fermentation broth. The solid-liquid mixture or system is preferably an aqueous system. Alternatively, the method may be known as separation or purification.

In the disclosure, the methods can include centrifugation to separate the biomass from the suspension. Further, the suspension may be pre-treated prior to any centrifugation. Advantageously, embodiments of the disclosure can increase sedimentation rate of the non-dissolved particles not by just few folds, but substantially by several orders of magnitude, e.g. 1000-10000-fold, compared to centrifuging without pre-treatment. Further, a clear supernatant (e.g. solution) containing the dissolved organic molecules can be produced.

In certain implementations, the suspension may be pre-treated by pH adjustment. In certain implementations, the suspension may be pre-treated by dilution. In certain implementations, the suspension may be pre-treated by heating. In certain variations, two or three of the disclosed pre-treatments can be performed. All pre-treatment disclosed below can be performed prior to centrifuging.

In particular, embodiments of the disclosure can be used for the separation of a liquid, preferably aqueous, phase containing human milk oligosaccharides (HMOs) from a suspension such as a fermentation broth. However, the particular separated material is not limiting, and the disclosure can be used for the separation/purification of other industrially useful organic compounds originating from a fermentation process or enzymatic reaction from biomass.

Advantageously, the pre-treatment can be performed without adding a flocculation agent (e.g. an agent that accelerates the process such as the sedimentation rate such as inorganic polyvalent metal salts or charged polymers (e.g. polyethyleneimine (PEI) or chitosan)). Thus, no flocculation agent may be used in any or all embodiments of the disclosure.

Typical centrifuging of the solution without embodiments of the disclosed pre-treatments required long centrifugation (residence) time at high g-force in lab-scale trials with unspecified yields and unspecified efficacy of removal of proteins and other biomolecules and unspecified degree of clarification, such as by OD600 measurements (optical density measured at a wavelength of 600 nm with a spectrophotometer). Thus, the known processes could not be used to effectively commercialize manufacturing, as only lab-scale operations could be performed.

However, through the use of embodiments of the disclosed pre-treatment methods, a number of advantages have been produced, such as 1) a substantially higher throughput is achieved; 2) a better yield of the dissolved organic compound, e.g. HMO, is achieved due to dilution and heat treatment, both of which minimize the biomass/supernatant ratio and facilitate extraction of product from biomass; 3) fine particle removal after pre-treatment is much more efficient as quantified by OD600; 4) biomolecule removal is also more efficient (e.g. proteins due to denaturation and precipitation at lower pH and heat treatment); 5) higher throughput, i.e. higher flux could be achieved in the subsequent optional membrane filtration step such as microfiltration or ultrafiltration.

Further, embodiments of the disclosure have shorter step durations, smaller and less complex equipment, less cleaning chemicals, and require shorter and less frequent cleaning.

### Human Milk Oligosaccharides (HMOs)

Human milk oligosaccharides (HMOs, human milk glycans) are complex carbohydrates that can be found in high concentrations in, for example, human breast milk (see e.g. Urashima et al .: Milk Oligosaccharides. Nova Science Publisher (2011); Chen Adv. Carbohydr. Chem. Biochem. 72, 113 (2015)). The HMOs have a core structure comprising a lactose unit at the reducing end that can be elongated by one or more β-N-acetyl-lactosaminyl and/or one or β-more lacto-N-biosyl units, and which core structure can be substituted by an α L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose | (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-fucopentaose VI (LNFP-VI), lacto-N-difucohexaose II (LNDFH-II), fucosyl-lacto-N-hexaose I (FLNH-I), fucosyl-para-lacto-N-hexaose | (FpLNH-I), fucosyl-para-lacto-N-neohexaose II (F-pLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

In one embodiment, the method of invention is suitable to purify acidic HMOs present in the suspension. If the suspension is a fermentation broth, the HMO is preferably 3′-SL, 6′-SL, LST a, LST b, LST c or DSLNT, more preferably, 3′-SL or 6′-SL. If the suspension is an enzymatic reaction milieu, the HMO is preferably LST a, fucosyl-LST a (FLST a), LST b, fucosyl-LST b (FLST b), LST c, fucosyl-LST c (FLST c) or DSLNT.

In one embodiment, the method of invention is suitable to purify neutral HMOs present in the suspension. If the suspension is a fermentation broth, the neutral HMO, as the main product of the fermentation is a trisaccharide, a tetrasaccharide, a pentasaccharide or a hexasaccharide neutral HMO, preferably a tri-, tetra- or pentasaccharide neutral HMO. A fermentation broth of any tri- to hexasaccharide neutral HMO may contain di- to heptasaccharide by-products, some them may be HMOs themselves. Typical trisaccharide neutral HMO fermentation broths are those of 2′-FL or 3-FL; typical tetrasaccharide neutral fermentation broths are those of LNT, LNnT or DFL; typical pentasaccharide neutral fermentation broths are those of LNFP-I, LNFP-II, LNFP-III, LNFP-V or LNFP-VI.

Preferably, the HMO in the method of invention is 2′-FL, 3-FL, DFL, LNT, LNnT, LNFP-I, LNFP-II, LNFP-III, LNFP-V or LNFP-VI.

### Method of Purification

Biomass removal (e.g. purification or solid-liquid separation) is one of the first steps for the isolation of industrially useful organic compounds, e.g. HMOs, from a suspension such as a fermentation broth or an enzymatic reaction milieu. In some implementations, biomass removal can be performed by centrifugation. Removal of biomass originating from microorganisms/cells or enzymes/proteins by centrifugation usually requires high g-force (i.e. RCF>10000 g; relative centrifugal force, the acceleration in a centrifuge normalized to Earth's gravity) and/or long residence time (e.g. >10 min) due to very small particle size and presence of even smaller cell fragments and other suspended solids.

Solid-liquid separation accelerated by centrifugation has been successfully applied in lab-scale for product separation from fermentation broths or enzymatic reaction milieu typically at high RCF>10000 g. However, when applied to industrial scale, it faces many general limitations, including complicated processes, heavy capital investment and high energy consumption. Acceleration of the large size rotor with the liquid feed to the required high velocity or high RCF requires not only high energy consumption but restrict the limit for RCF due to much lower tolerance for the generated internal wall pressure compared to small size lab centrifuges that could run up to RCF=1,000,000 g with low risk of a catastrophic failure. Therefore, fermentation broths or enzymatic reaction milieu are usually processed by micro- or ultrafiltration as a widely applied alternative for biomass removal. However, this approach also requires sophisticated and expensive equipment and highly robust membranes against the feed with high suspended solid content. In addition, it associates with high energy consumption due to high pressure drop associated with the required high cross-flow velocity and high viscosity. Another issue is unavoidable time-dependent membrane fouling which could substantially decrease the permeate flux.

In order to make separation by centrifugation of fermentation broths or enzymatic reaction milieu economically feasible in industrial scale, the broth or the enzymatic reaction milieu must be pre-conditioned, ideally with the change of all the parameters that affect the sedimentation rate, i.e. lower viscosity, higher density difference between suspended solid particles and media and bigger particle size, which would allow to run the process at lower g-force and shorter residence time. A typical approach to enlarge particles is flocculation where cell or other particles are aggregated in the presence of a flocculation agent. The approach is quite straight-forward and involves addition of a flocculant such as inorganic polyvalent metal salts or charged polymers such as polyethyleneimine (PEI) or chitosan to the broth prior to sedimentation or centrifugation. Although flocculation is widely used in e.g. waste water treatment, its application for food-grade products could be prohibitive not only due to high cost of flocculants but also due to potential toxicity and regulatory requirements.

As will be discussed in detail below, embodiments of the disclosure can greatly improve biomass removal speed through the continuous centrifugation of a fermentation broth or a enzymatic reaction milieu which is pre-treated prior to the centrifuging. Embodiments of the disclosure have advantageously found that pre-treatment of the broth or the enzymatic reaction milieu by one or more of pH-adjustment, dilution and heat can increase sedimentation rate not by just few folds, but substantially by several orders of magnitude, i.e. by 1000-10000-fold. For example, settling velocities of E. coli fermentation broths were found typically to be in the range of 0.002-0.01 mm/h at normal gravity, which could be accelerated upon pre-treatment up to 50 mm/h. In certain implementations, any one, two, or three of pH-adjustment, dilution, and heat can be performed for the pre-treatment.

Also, the pre-treatment can minimize or eliminate biomolecule content, such as cell fragment, polynucleotide, protein or other biopolymers, in the supernatant, and can minimize residual suspended solids that could be estimated by OD600, and can increase the yield of the dissolved organic compound of interest, e.g. an HMO product, in the supernatant after just a single centrifugation step.

Further, the centrifugation can either replace ultrafiltration as biomass removal step or increase the ultrafiltration throughput substantially making the overall process more economical and efficient.

In addition, other parameters of production process such as high product yield, low residual suspended solids, and low protein content are substantially improved as well. These surprising results could be rationalized to some extent by simultaneous change of all the physical parameters that influence the sedimentation rate: dilution reduces the density of the media and lower the viscosity; pH adjustment can reduce the surface charge of particles facilitating their self-flocculation; further heat treatment especially at low pH can denature and precipitate biomolecules such as proteins and DNAs that contribute a lot to the high broth viscosity. Also, these charged biopolymers after unfolding by pH and heat-treatment could serve as endogenous flocculation agents inducing particle enlargement that is apparent to the naked eye.

Accordingly, embodiments of the disclosure can substantially accelerate throughput, thereby making the industrial bio-mass removal process economically feasible.

### Suspension containing biomass

The disclosed methodology can be used in conjunction with a suspension, generally. A suspension is a heterogeneous solid-liquid mixture or system wherein the solid phase or component is distributed in a liquid phase. The liquid phase advantageously comprises water (i.e. aqueous suspension). In an aqueous suspension, the solid phase or component can be a biomass (suspended, precipitated or insoluble materials originating from fermentation cells, like intact cells, disrupted cells, cell fragments, proteins, protein fragments, polysaccharides, etc) or proteins that may be partially or fully denatured and/or precipitated in an enzymatic reaction mixture. Preferably, the liquid phase of the suspension contains dissolved organic molecule(s) that is/are (a) product(s) of a fermentative process (as a partially secreted secondary metabolite) or of an in vitro enzymatic reaction.

A particular suspension can be discussed herein in conjunction with HMOs as dissolved organic molecules. For example, the suspension can be a fermentation broth. Accordingly, the biomass can be a biomass originated from bacterial or fungal cells.

Advantageously, the use of a modified E.coli as bacterium within a bacterial fermentation broth can produce HMOs, such as discussed below. For example, the suspension can be produced by genetically modified E.coli capable of producing HMOs.

Preferably, the reaction milieu in which an HMO has been produced is a fermentation broth. The fermentation broth typically contains, besides the HMO of interest as main compound for the production of which a genetically modified microorganism, preferably an E. coli, has been suitably designed, carbohydrate by-products or contaminants such as carbohydrate intermediates in the biosynthetic pathway of the HMO of interest from a precursor, typically lactose, preferably exogenously added lactose, and/or those as a result of a deficient, defective or impaired glycosylation during the biosynthetic pathway, and/or those as a result of rearrangement or degradation under the cultivation condition or post-fermentative operations, and/or unconsumed precursor added in excess during the fermentation. Further, the fermentation broth can contain cells, proteins, protein fragments, DNA, caramelized by-products, minerals, salts, organic acids, endotoxins and/or other charged molecules and metabolites.

A broth can include a cell of a microorganism, such as a bacterial or fungi cell, e.g. an E.coli cell, which has been genetically manipulated to include at least one alteration in its DNA sequence. The bacteria can undergo genetic alteration that can result in a change in the original characteristics of the wild type cell, e.g. the modified cell is able to perform additional chemical transformation due to the introduced new genetic material that encodes the expression of an enzymes not being in the wild type cell, or is not able to carry out transformation like degradation due to removal of gene/genes (knockout). A genetically modified cell can be produced in a conventional manner by genetic engineering techniques that are well-known to those skilled in the art.

Further, a microbial fermentation can produce an HMO from an internalized carbohydrate precursor, preferably means a cell of a microorganism, such as a bacterium or fungi (e.g. yeast), preferably a bacterium, more preferably an E. coli, which is genetically manipulated (vide supra) to comprise one or more endogenous or recombinant genes encoding one or more glycosyl transferase enzymes that are able to transfer the glycosyl residue of an activated sugar nucleotide to an internalized acceptor molecule and necessary for the synthesis of said neutral HMO, a biosynthetic pathway to produce the corresponding activated sugar nucleotide donor(s) suitable to be transferred by said glycosyl transferase to a carbohydrate precursor (acceptor) and a mechanism of internalization of a carbohydrate precursor (acceptor) from the culture medium into the cell where it is glycosylated to produce the neutral HMO of interest. The glycosyl transferases are selected from ß-1,3-N-acetylglucosaminyl transferase, ß-1,6-N-acetylglucosaminyl transferase, ß-1,3-galactosyl transferase, ß-1,4-galactosyl transferase, a-1,2-fucosyl transferase, a-1,3-fucosyl transferase, a-1,4 fucosyl transferase, a-2,3 sialyl transferase, a-2,6 sialyl transferase. The corresponding activated sugar nucleotides are UDP-Gal, UDP-GIcNAc, GDP-Fuc and CMP-sialic acid.

During the fermentation of the fermentation broth, a biomass is created. The biomass can refer to suspended, precipitated or insoluble materials originating from fermentation cells, like intact cells, disrupted cells, cell fragments, proteins, protein fragments, polysaccharides. In order to remove the biomass from the suspension (broth), and in particular to produce purified HMOs, a number of processes can be taken as discussed herein.

### Pre-Treatment

One or more exemplary methods can allow for the pre-treatment of a suspension, such as those described herein. However, the pre-treatment can be used for other suspension as well and should not be so limited to those described in detail herein.

As will be discussed in further detail below, the pre-treatment of the suspension can include pH adjustment, and/or dilution, and/or heat treatment. In certain implementations, all three of pH adjustment, dilution, and heat treatment can be performed. In alternative embodiments, pH adjustment and dilution can be performed. In alternative embodiments, pH adjustment and heat treating can be performed. In alternative embodiments, heat treating and dilution can be performed.

Advantageously, a combination of a plurality of pre-treatment methods can provide an improved synergistic effect not found in individual pre-treatments.

All pre-treatment can be performed prior to centrifuging, or otherwise separating the HMOs, of the pre-treated suspension. In certain embodiments, one or more pre-treatment steps can occur during the centrifuging. For example, between steps in a multi-step centrifuging. Alternatively, the centrifuging vessel can heat the suspension during centrifuging.

Advantageously, the pre-treatment can increase the settling velocity of the solid particles (biomass) in the suspension (broth) by 100-20000 fold making the biomass separation by centrifugation much more efficient and thus applicable in industrial scale. In addition to settling velocity, at least 3 other parameters are substantially improved due to pre-treatment:
1) The yield of the dissolved organic compound in the liquid phase (supernatant), preferably HMO, could be increased to >90% compared to only around 50-70% without pre-treatment after single centrifugation;
2) The protein and DNA content in supernatant could be reduced by around 10-fold;
3) The residual suspended solid content is also substantially reduced to OD600<0.1, such as OD600<0.05, e.g. to OD600 around 0.024, by centrifugation at low RCF=around 3000 g for 3 min compared to OD600>1 at around 10000 g/10 min for the untreated broth.

### Pre-treatment via pH Adjustment

In one or more exemplary methods, the pH of the suspension (e.g. broth, fermentation broth) can be adjusted prior to/during centrifuging.

In many cases utilizing fermentation broths, the pH of the suspension is in the range of 6-7. For example, the pH may be at 6.1, 6.2, 6.3, 6.4, 6.5, 6.6, 6.7, 6.8, or 6.9. However, other suspensions may have different starting pHs, such as below 6 and above 7. The particular starting pH of the suspension is not limiting.

Advantageously, reducing the pH of the suspension, therefore making it acidic, prior to centrifuging may improve sedimentation rate, and thus speed and yield processes. Further, a flocculation agent may be avoided as the pH adjustment may serve as an endogenous flocculation process.

The pH of the suspension may be adjusted through any number of methods. For example, an acid can be incorporated into the suspension to reduce the pH of the suspension. The acid can be an organic acid. In other embodiments, the acid can be an inorganic acid.

One example acid that can be used is sulfuric acid, such as having the formula H₂SO₄. For example, the acid could be 20% H₂SO₄. However, other acids can be used as well, and the particular acid used is not limiting. For example, the acid can be selected from one or more of H₂SO₄, HCl, H₃PO₄, formic acid, acetic acid, and citric acid. Each of these acids can be used alone or in combination with any other acids.

Through the pH adjustment, the suspension may be brought down to a pH of between 2 and 5, in particular between 3 and 4. For example, after the pH adjustment pre-treatment, the suspension may have a pH of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. In certain implementations, after the pH adjustment pre-treatment, the suspension may have a pH of greater than 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, or 4.9. In some implementations, after the pH adjustment pre-treatment, the suspension may have a pH of less than 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

In one embodiment, after adjusting the pH of the suspension acidic, the pre-treated suspension is cooled down, e.g. to 5-15° C., and stored for 1-15 days before centrifuging.

Advantageously, the pH adjustment of the suspension can reduce a surface charge of particles (e.g. those of the biomass). Therefore, the pH adjustment can facilitate self-flocculation of the biomass, and thus centrifuging.

In one or more exemplary methods, the pH adjustment can be performed alone, with the dilution, with the heat treatment, or with both the heat treatment and the dilution.

### Pre-treatment via Dilution

In one or more exemplary methods of the disclosed methods, the suspension (e.g. broth, fermentation broth) can be diluted prior to/during centrifuging.

In certain embodiments, the suspension can be diluted so that the mass of the diluted suspension is between 1.1 and 10 times of the original mass of the suspension, preferably between 1.5 and 10, more preferably between 2 and 4, more preferably between 2.5 and 3.5. For example, the suspension may be diluted by 1.1, 1.2, 1.3, 1.4, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 times the original mass of the suspension. In some embodiments, the suspension may be diluted by less than 2.0, 2.5, 3.0, 3.5, 4.0, 4.5, 5.0, 5.5, 6.0, 6.5, 7.0, 7.5, 8.0, 8.5, 9.0, 9.5, or 10.0 times the original mass of the suspension. In some embodiments, the suspension may be diluted by greater than 1.5, 2.0, 2.5, 3.0, 3.5, 4.0, 4.5, 5.0, 5.5, 6.0, 6.5, 7.0, 7.5, 8.0, 8.5, 9.0, or 9.5 times the original mass of the suspension.

The dilution can be performed preferably with water. The water may be tap water, de-ionized water or distilled water.

In certain implementations, dilution can lower the density of supernatant solution and lower the viscosity. Another advantage of dilution is to increase the yield of the HMO product available in the supernatant. For example, without dilution, only around 50-70% of the HMO produced by fermentation go to the supernatant.

In certain implementations, after dilution the suspension may have a dilution ratio from about 2 to 10, preferably between 2 and 8, more preferably between 2 and 4, more preferably between 2.5 and 3.5. The dilution ratio is a ratio between the final volume of the suspension (i.e. after dilution) and the initial volume of the suspension (i.e. before dilution). For example, the suspension may be diluted by 2.0, 2.5, 3.0, 3.5, 4.0, 4.5, 5.0, 5.5, 6.0, 6.5, 7.0, 7.5, 8.0, 8.5, 9.0, 9.5, or 10.0 times the original volume of the suspension.

Preferably, the suspension may be diluted to achieve a particular bio-wet-mass (BWM). BWM is a ratio between the isolated wet solid pellet after centrifugation vs initial broth mass. To determine the BWM, a sample is taken from the stirred suspension and centrifuged with a relative centrifugal force (RCF) of around 10000 g until a clear supernatant is obtained (about 10 min). The supernatant is decanted and the mass of the solid wet pellet is weighted. BWM is the percent ratio of the solid wet pellet to the weight of the sample taken from the suspension. Typical suspensions have a BWM of 25-35%, or all the way up to 50%, as determined under the conditions disclosed above. Thus, in some embodiments, the suspension may be diluted so that its BWM is less than 20, 15, 10 or 5%, preferably around 10-15% (the BWM after dilution is determined under identical conditions as before dilution). More preferably, this is achieved by a dilution with a dilution ratio of around 2.5-3.5, or so that the mass of the diluted suspension is between 2.5 and 3.5 of the original mass of the suspension. In some embodiments, the suspension may be diluted so that is BWM of the original suspension is reduced by 30-90%, for example the BWM is reduced to e.g. ⅔, ½, ⅓, ⅕ or 1/10 of the original BMW measured before dilution.

In some embodiments, dilution can be performed during a multiple-stage centrifugation, such as a 2-stage centrifugation. For example, the water may be added after the first centrifugation, but before the second centrifugation.

In one or more exemplary methods, the dilution can be performed alone, with the pH adjustment, with the heat treatment, or with both the heat treatment and the pH adjustment.

### Pre-treatment via Heat Treatment

In one or more exemplary methods, the suspension can be heat treated (e.g. heat adjusted, temperature treated, change of temperature) prior to/during centrifuging.

In certain embodiments of the disclosure, the suspension can be pre-treated by heating the suspension up to a temperature of between 20 and 120° C., preferably 45 and 120° C., preferably between 60 and 90° C., more preferably between 60° C. and 80° C. For example, the suspension may be heated to a temperature of 20, 25, 30, 35, 40, 45, 50, 55, 60, 65, 70, 75, 80, 85, 90, 95, 100, 105, 110, 115, or 120° C. In some embodiments, the suspension may be heated to a temperature of greater than 20, 25, 30, 35, 40, 45, 50, 55, 60, 65, 70, 75, 80, 85, 90, 95, 100, 105, 110, 115, or 120° C. In some embodiments, the suspension may be heated to a temperature of less than 20, 25, 30, 35, 40, 45, 50, 55, 60, 65, 70, 75, 80, 85, 90, 95, 100, 105, 110, 115, or 120ºC.

The heat treatment can be performed through known methods, such as the use of an oven, a burner, water bath, oil bath, heating mantle or other heating systems. Preferably, the heat treatment is performed by means of an industrial heater using one or a combination of the following heat transfer methods: conductive, convective, radiation. Such heaters can be e.g. circulation heaters, duck heaters, immersion heaters, heating with steam or heating through heating mantle.

In some embodiments, the suspension can be held at the heat treatment temperature for a particular time. For example, the suspension can be held at any of the above recited heating temperatures for 1, 2, 3, 4, 5, 10, 15, 20, 25, or 30 minutes. In some embodiments, the suspension can be held at any of the above recited heating temperatures for greater than 1, 2, 3, 4, 5, 10, 15, 20, 25, or 30 minutes. In some embodiments, the suspension can be held at any of the above recited heating temperatures for less than 2, 3, 4, 5, 10, 15, 20, 25, or 30 minutes.

In certain embodiments, the suspension is pre-treated by directly heating to any of the above-listed temperatures. In other implementations, the suspension can include stepwise heating, where the suspension can be heated to a first temperature for a first amount time. The suspension can then be heated to a second temperature for a second amount of time. The first temperature can be less than the second temperature. The first temperature can be greater than the second temperature (e.g. cooled instead of heated). The first amount of time can be greater than the second amount of time. The first amount can be less than the second amount of time. The first temperature can be any of the above-described temperatures. The second temperature can be any of the above-described temperatures. The first amount of time can be any of the above-described times. The second amount of time can be any of the above-described times.

Advantageously, the heat treatment can denature portions of the biomass, such as proteins and DNAs. Further, the heat treatment can precipitate biomolecules, such as proteins and DNAs. This can contribute to lowering the high initial broth viscosity and thus increase the settling velocity of the solid particles (biomass) in the suspension (broth), making the biomass separation by centrifugation much more efficient.

In one or more exemplary methods, the heat treatment can be performed alone, with the dilution, with the pH adjustment, or with both the pH adjustment and the dilution.

### Pre-treatment via pH Adjustment and Dilution

In one or more exemplary methods, both a pH adjustment and a dilution of the suspension may be performed. The combination of the two methodologies may provide synergistic effects that allow for a clearer supernatant and ease of centrifuging. For example, pH adjustment can reduce surface charge and when combined with dilution should provide for a better HMO yield.

In one or more exemplary methods, the pH adjustment of the suspension can be performed first, as discussed in detail above. Once the pH has been adjusted as desired, the suspension may be diluted to the desired level, as discussed in detail above. If the dilution affects the pH, the pH may be re-balanced in order to maintain the proper pH.

In one or more exemplary methods, the dilution of the suspension can be performed first, as detailed above. Once the suspension has been diluted to the desired level, the suspension can then undergo pH adjustment. Thus, the final diluted suspension may undergo pH adjustment which may require more acid to be used as compared to pH adjustment alone due to the higher volume of suspension.

In one or more exemplary methods, the dilution and the pH adjustment can occur at the same time (e.g. generally at the same time, concurrently, simultaneously). For example, water can be added into the suspension, and while this occurs acid may also be added. Alternatively, acid may be added first and water can be added at around the same time. In one or more exemplary methods, the pH adjustment and dilution can occur in repeating steps. For example, some acid may be added, then some water, which can be repeated until a desired pH and dilution is achieved.

Both the pH adjustment and dilution may occur prior to centrifuging or during centrifuging.

### Pre-treatment via pH Adjustment and Heating

In one or more exemplary methods, both a pH adjustment and heating of the suspension may be performed. The combination of the two methodologies may provide synergistic effects that allow for a clearer supernatant and ease of centrifuging. For example, pH adjustment can reduce surface charge and when combined with denaturing of the heating a better HMO yield can be achieved.

In one or more exemplary methods, the pH adjustment of the suspension can be performed first as discussed in detail above. Once the pH has been adjusted as desired, the suspension may be heated to the desired temperature(s), as discussed in detail above. If the heating affects the pH, the pH may be re-balanced in order to maintain the proper dilution.

In one or more exemplary methods, the heating of the suspension can be performed first, as detailed above. Once the suspension has been heated to the desired temperature(s), the suspension can then undergo pH adjustment. The amount of acid required in combination with the heat treatment may change based on pH adjustment alone.

In one or more exemplary methods, the heating and the pH adjustment can occur at the same time (e.g. generally at the same time, concurrently, simultaneously). For example, a vessel containing the suspension may be heated in one or more of the methods discussed above. While the vessel is heating up, acid may be added to the suspension in order to adjust the pH of the suspension.

Both the pH adjustment and dilution may occur prior to centrifuging or during centrifuging.

### Pre-treatment via Dilution and Heating

In one or more exemplary methods, both a dilution and heating of the suspension may be performed. The combination of the two methodologies may provide synergistic effects that allow for a clearer supernatant and ease of centrifuging. For example, the denaturing of proteins achieved by heating may allow for easier separation from dilution, which can lead to a better HMO yield.

In one or more exemplary methods, the dilution of the suspension can be performed first as discussed in detail above. Once the suspension has been diluted, the suspension may be heated to the desired temperature(s), as discussed in detail above. If the heating affects the dilution, such as the loss of material through evaporation, further water can be added in order to maintain the proper dilution.

In one or more exemplary methods, the heating of the suspension can be performed first, as detailed above. Once the suspension has been heated to the desired temperature(s), water can be added into the suspension. The water may be at the same temperature as the heated suspension, or may be at a lower or higher temperature.

In one or more exemplary methods, the heating and the dilution can occur at the same time (e.g. generally at the same time, concurrently, simultaneously). For example, a vessel containing the suspension may be heated in one or more of the methods discussed above. While the vessel is heating up, water may be added to the suspension in order to adjust the dilution of the suspension. Alternatively, dilution and heating can occur at the same time by adding hot water pre-heated to a temperature higher than the target temperature of the diluted suspension after adding into a cold suspension.

Both the heating and dilution may occur prior to centrifuging or during centrifuging.

### Pre-treatment via Heating, pH Adjustment, and Dilution

In one or more exemplary methods, all three pre-treatments of a dilution, a heating, and a pH adjustment of the suspension may be performed. The combination of the three methodologies may provide synergistic effects that allow for a clearer supernatant and ease of centrifuging. Thus, higher yields at faster times can be achieved. The combination of the three pre-treatments can have a greater impact than the addition of all pre-treatments individually.

In one or more exemplary methods, the pH adjustment of the suspension can be performed first as discussed in detail above. Once the pH has been adjusted as desired, the suspension may be diluted to the desired level, as discussed in detail above. If the dilution affects the pH, the pH may be re-balanced in order to maintain the proper pH. Once the dilution has been performed, the suspension may be heated to the desired temperature(s), as discussed in detail above. If the heating affects the pH and/or the dilution, the pH and/or the dilution may be re-balanced in order to maintain the proper pH levels and/or dilution levels.

In one or more exemplary methods, the pH adjustment of the suspension can be performed first as discussed in detail above. Once the pH adjustment has been performed, the suspension may be heated to the desired temperature(s). If the heating affects the pH, the pH may be re-balanced in order to maintain the proper pH levels. Once the heating has been performed, the suspension may be diluted to the desired level. If the dilution affects the pH, the pH may be re-balanced in order to maintain the proper pH. The water (e.g. for dilution) may be at the same temperature as the heated suspension, may be at a lower temperature or at higher temperature.

In one or more exemplary methods, the heating of the suspension can be performed first, as detailed above. Once the suspension has been heated to the desired temperature(s), the suspension can then undergo pH adjustment. The amount of acid required in combination with the heat treatment may change based on pH adjustment alone. Once the heating and pH adjustment have been performed, the suspension may be further diluted. The water may be at the same temperature as the heated suspension, may be at a lower temperature or at higher temperature. If need be after dilution, the pH can be further adjusted to a desired level.

In one or more exemplary methods, the heating of the suspension can be performed first, as detailed above. Once the suspension has been heated to the desired temperature(s), the suspension can then undergo dilution. The water may be at the same temperature as the heated suspension, may be at a lower temperature or at higher temperature. Once the heating and dilution have been performed, the suspension may undergo pH adjustment. The amount of acid required in combination with the heat treatment and dilution may change based on pH adjustment alone.

In one or more exemplary methods, the dilution of the suspension can be performed first as discussed in detail above. Once the suspension has been diluted, the suspension may be heated to the desired temperature(s). If the heating affects the dilution, such as the loss of material through evaporation, further water can be added in order to maintain the proper dilution. After the heating, the suspension may undergo pH adjustment. The amount of acid required in combination with the heat treatment and dilution may change based on pH adjustment alone.

In one or more exemplary methods, the dilution of the suspension can be performed first as discussed in detail above. Once diluted, the suspension may undergo pH adjustment. The amount of acid required in combination with the heat dilution may change based on pH adjustment alone. After the pH adjustment, the suspension may be heated. Adjustments can be made to the suspension during the heating to maintain pH and dilution levels.

In one or more exemplary methods, the heating and the dilution can occur at the same time (e.g. generally at the same time, concurrently, simultaneously). For example, a vessel containing the suspension may be heated in one or more of the methods discussed above. While the vessel is heating up, water may be added to the suspension in order to adjust the dilution of the suspension. After the heating and dilution, the pH of the suspension then may be adjusted. Alternatively, the pH of the suspension may be adjusted prior to the concurrent heating and dilution.

In one or more exemplary methods, the heating and the pH adjustment can occur at the same time (e.g. generally at the same time, concurrently, simultaneously). For example, a vessel containing the suspension may be heated in one or more of the methods discussed above. While the vessel is heating up, acid may be added to the suspension in order to adjust the pH of the suspension. After the heating and the pH adjustment, the suspension may be diluted. Alternatively, the suspension may be diluted prior to a concurrent heating and pH adjustment.

In one or more exemplary methods, the dilution and the pH adjustment can occur at the same time (e.g. generally at the same time, concurrently, simultaneously). For example, water can be added into the suspension, and while this occurs acid may also be added. Alternatively, acid may be added first, and water can be added at around the same time. In one or more exemplary methods, the pH adjustment and dilution can occur in repeating steps. For example, some acid may be added, then some water, which can be repeated until a desired pH and dilution is achieved. After the dilution and the pH adjustment, the suspension may then be heated. Alternatively, the suspension may be heated prior to the concurrent dilution and pH adjustment.

In one or more exemplary methods, the dilution, the pH adjustment, and the heating can occur at the same time (e.g. generally at the same time, concurrently, simultaneously). Thus, all three pre-treatments can occur at the same time.

The heating, the pH adjustment, and the dilution may occur prior to centrifuging or during centrifuging.

### Centrifugation

After any or all of the above pre-treatments, it is necessary to separate the biomass from the suspension. One such method is centrifuging, which can have advantages over other processes such as ultrafiltration, which can take a significant amount of time. The centrifuging can be lab scale or, advantageously over previous centrifuging methods, commercial scale (e.g. industrial scale, full production scale).

In some embodiments, a multi-step centrifugation can be used. For example, a series of 2, 3, 4, 5, 6, 7, 8, 9, or 10 centrifugation steps can be performed. In other implementations, the centrifugation may be a single step.

Centrifugation provides a quick biomass-removal compared to lengthy ultrafiltration both on lab and full production scale with a long clean-in-place (CIP) procedure, requirement of expensive ceramic membranes and high size/high foot-print area.

Biomass separation is then achieved by a more efficient and economical way compared to ultrafiltration. The current biomass removal by industrial continuous ultrafiltration has low permeate flow rate, that requires high residence time (>1 h), high membrane area associated with equipment high foot-print area, high energy consumption by powerful cross-flow pumps, has performance that is broth-dependent and sometime unpredictable, requires long CIP (5 h) every 24 h of operation or less, has a CapEx which is very high both for UF unit and ceramic membranes, and has a high size/footprint.

In certain embodiments, Sedicanter® centrifuge designed and manufactured by Flottweg can be used.

The particular type of centrifuge is not limiting, and many types of centrifuges can be used. The centrifuging can be a continuous process. In some embodiments, the centrifuging can have feed addition. For example, the centrifuging can have a continuous feed addition. In certain embodiments, the centrifuging can include a solid removal, such as a wet solid removal. The wet solid removal can be continuous in some implementations, and periodic in other implementations.

For example, a conical plate centrifuge (e.g. disk bowl centrifuge or disc stack separator) can be used. The conical plate centrifuge can be used to remove solids (usually impurities) from liquids or to separate two liquid phases from each other by means of a high centrifugal force. The denser solids or liquids which are subjected to these forces move outwards towards the rotating bowl wall while the less dense fluids move towards the centre. The special plates (known as disc stacks) increase the surface settling area which speeds up the separation process. Different stack designs, arrangements and shapes are used for different processes depending on the type of feed present. The concentrated denser solid or liquid can then be removed continuously, manually or intermittently, depending on the design of the conical plate centrifuge. This centrifuge is very suitable for clarifying liquids that have small proportion of suspended solids.

The centrifuge works by using the inclined plate setter principle. A set of parallel plates with a tilt angle θ with respect to horizontal plane is installed to reduce the distance of the particle settling. The reason for the tilted angle is to allow the settled solids on the plates to slide down by centrifugal force so they do not accumulate and clog the channel formed between adjacent plates.

This type of centrifuge can come in different designs, such as nozzle-type, manual-cleaning, self-cleaning, and hermetic. The particular centrifuge is not limiting.

Factors affecting the centrifuge include disk angle, effect of g-force, disk spacing, feed solids, cone angle for discharge, discharge frequency, and liquid discharge.

Alternatively, a solid bowl centrifuge (e.g. a decanter centrifuge) can be used. This is a type of centrifuge that uses the principle of sedimentation. A centrifuge is used to separate a mixture that consists of two substances with different densities by using the centrifugal force resulting from continuous rotation. It is normally used to separate solid-liquid, liquid-liquid, and solid-solid mixtures. One advantage of solid bowl centrifuges for industrial uses is the simplicity of installation compared to other types of centrifuge. There are three design types of solid bowl centrifuge, which are conical, cylindrical, and conical-cylindrical.

Solid bowl centrifuges can have a number of different designs, any of which can be used for the disclosed method. For example, conical solid bowl centrifuges, cylindrical solid bowl centrifuges, and conical-cylindrical bowl centrifuges can be used.

With the help of helical screw conveyor, solid bowl centrifuges separate two substances with different densities by the centrifugal force formed under fast rotation. Feed slurry enters the conveyor and is delivered into the rotating bowl through discharge ports. There is a slight speed difference between the rotation of conveyor and bowl, causing the solids to convey from the stationary zone where the wastewater is introduced to the bowl wall. By centrifugal force, the collected solids move along the bowl wall, out of the pool and up the dewatering beach located at the tapered end of the bowl. At last the solids separated go to solid discharge while the liquids go to liquid discharge. The clarified liquid flows through the conveyor in the opposite direction through adjustable overflow parts.

The centrifuging can be performed at a number of speeds and residence times. For example, the centrifuging can be performed with a relative centrifugal force (RCF) of 20000 g, 15000 g, 10000 g, or 5000 g. In some embodiments, the centrifuging can be performed with a relative centrifugal force (RCF) of less than 20000 g, 15000 g, 10000 g or 5000 g. In some embodiments, the centrifuging can be performed with a relative centrifugal force (RCF) of greater than 20000 g, 15000 g, 10000 g or 5000 g.

In some embodiments, the centrifuging can be characterized by working volume. In some embodiments, the working volume can be 1, 5, 10, 15, 20, 50, 100, 300, or 500 l. In some embodiments, the working volume can be less than 1, 5, 10, 15, 20, 50, 100, 300, or 500 l. In some embodiments, the working volume can be greater than 1, 5, 10, 15, 20, 50, 100, 300, or 500 l.

In some embodiments, the centrifuging can be characterized by feed flow rate. In some embodiments, the feed flow rate can be 100, 500, 1000, 1500, 2000, 5000, 10000, 20000, 40000, or 100000 l/hr. In some embodiments, the feed flow rate can be greater than 100, 500, 1000, 1500, 2000, 5000, 10000, 20000, 40000, or 100000 l/hr. In some embodiments, the feed flow rate can be less than 100, 500, 1000, 1500, 2000, 5000, 10000, 20000, 40000, or 100000 l/hr.

The amount of time spent centrifuging (e.g. residence time) can vary as well. For example, the residence time can be 0.1, 0.2, 0.5, 1, 2, 3, 4, 5, 6, 7, 8, 9, or 10 minutes. In some embodiments, the residence time can be greater than 0.1, 0.2, 0.5, 1, 2, 3, 4, 5, 6, 7, 8, 9, or 10 minutes. In some embodiments, the residence time can be less than 0.1, 0.2, 0.5, 1, 2, 3, 4, 5, 6, 7, 8, 9, or 10 minutes.

### Supernatant

The above described processes can allow for the formation of an improved supernatant. For example, the supernatant can have the HMOs, while the remaining material can be separated out. Thus, the supernatant can be a clear (e.g. clarified, generally clear, mostly clear) supernatant. The remaining material (which can cause a lack of clarity of the supernatant) can be, for example, a biomass, that is one or more of cells, cell fragments, small particles, and biomolecules. The biomolecules can be, for example, proteins, RNAs, and DNAs. Thus, if HMOs are desired, the supernatant can be a clarified supernatant containing purified HMOs.

As an example of clarity for a supernatant, a typical fermentation broth can have very high optical density at 600 nm (OD600), more than 10 relative to pure water. OD600 is usually measured with a spectrophotometer; dilution of the sample may be necessary if OD600 goes above 1 due to high amount of suspended particles (and thus the OD600 value is calculated with respect to the dilution). However, after the above-recited processes, the OD600 can move to below 1.0, 0.7, 0.5, 0.3, 0.2, 0.1, or 0.05, which is measured without dilution but relative to micro-filtered sample of the obtained supernatant. In some embodiments, the OD600 can move to above 1.0, 0.7, 0.5, 0.3, 0.2, 0.1, or 0.05. In some embodiments, the OD600 can move to 1.0, 0.7, 0.5, 0.3, 0.2, 0.1, or 0.05. When OD600 is less than 0.1, the suspended particles cannot be visually detected and the supernatant would look as a clear solution.

Further, the proteins in the supernatant can be reduced as well, providing further clarity. The proteins in the supernatant post centrifuging can be less than 3000, 2500, 2000, 1500, 1000, 500, 400, 300, 200, 100, or 50 mg/l. In some embodiments, the proteins in the supernatant post centrifuging can be greater than 3000, 2500, 2000, 1500, 1000, 500, 400, 300, 200, 100, or 50 mg/l. In some embodiments, the proteins in the supernatant post centrifuging can be 3000, 2500, 2000, 1500, 1000, 500, 400, 300, 200, 100, or 50 mg/l.

Further, advantageously embodiments of the disclosed methods can provide for a higher product yield as compared to previous methods. The clarified supernatant can have a product yield of 50, 55, 60, 65, 70, 75, 80, 85, 90, or 95%. The clarified supernatant can have a product yield of >50, 55, 60, 65, 70, 75, 80, 85, 90, or 95%. The clarified supernatant can have a product yield of <50, 55, 60, 65, 70, 75, 80, 85, 90, or 95%.

Any of the above supernatant properties can be produced through a single instance of centrifuging. Alternatively, it can be produced through multiple instances of centrifuging.

### Post-Processing for Purification

After producing the above-described supernatant, further post-processing steps can be taken. This can be done to, for example, isolate (e.g. obtain) HMOs, or other products, from the supernatant. A number of different methods are discussed below. It will be understood that the method can include any one of the disclosed post-processing steps. In alternative embodiments, the method can contain more than one of the disclosed post-processing steps, in any configuration of the steps.

In some embodiments, the post-processing method can include ultrafiltration (UF) or microfiltration (MF) of the clarified supernatant. For example, the filtration can use a membrane having a pore size <2 μm and >2 nm and/or MWCO>1000 Da or 10000 Da or above. The pore size or cut-of should be large enough to allow the product to pass though the pores of the membrane. Optionally, there can further be diafiltration, wherein the HMOs are collected in a permeate stream.

The ultrafiltration step is to separate the remaining part of the biomass after centrifugation. The UF permeate (UFP) is an aqueous solution containing the produced HMO. The UF (or MF) membrane could be composed of a polymeric or non-polymeric material. Preferably, the UF membrane is composed of a non-polymeric material, more preferably a ceramic material. The non-polymeric UF membrane tolerates high temperature if UF is carried out at that temperature. Also, the applicable flux of a non-polymeric membrane, advantageously a ceramic membrane, is usually higher than that of a polymeric UF membrane with identical or similar MWCO; an addition, a non-polymeric membrane, advantageously a ceramic membrane, is less prone to fouling or getting clogged. In industrial application, the regeneration of the UF membrane is an important cost and technical factor. A non-polymeric membrane, advantageously a ceramic membrane, allows to use harsh clean-in-place (CIP) conditions including caustic/strong acid treatment at high temperature (not applicable on polymeric membranes), which may be required when a fermentation stream with high suspended solid content is ultrafiltered. Furthermore, a non-polymeric membrane, advantageously a ceramic membrane, has longer life-time due to inertness and abrasion-resistance to solid particles that circulate at high cross-flow.

Any conventional ultrafiltration membrane, advantageously ceramic membrane, can be used having a molecular weight cut-off (MWCO) range between about 1 and about 1000 kDa, such as around 1-10, 10-1000, 5-250, 5-500, 5-750, 50-250, 50-500, 50-750, 100-250, 100-500, 100-750, 250-500, 250-750, 500-750 kDa, or any other suitable sub-ranges.

UF can be conducted at low temperature (around 5° C. to rt), at around room temperature or at elevated temperature, preferably at elevated temperature. The elevated temperature preferably does not exceed around 80° C.; suitable temperature ranges can be e.g. around 35-50, 35-80, 45-65, 50-65, 55-65 or 60-80° C. The UF step conducted at elevated temperature substantially reduces the total number of viable microorganisms (total microbial count) in the reaction milieu thus a sterile filtration step in the later phase of the method may not be necessary. Furthermore, it reduces the amount of soluble proteins due to more effective denaturation and precipitation which increases the efficacy of residual protein removal in ion exchange treatment steps (as an optional step later).

The ultrafiltration step can be applied in dead-end or cross-flow mode. Yet in one embodiment, the ultrafiltration can be combined with diafiltration.

In some embodiments, the post-processing method can include nanofiltration (NF). This can be performed with, for example, a membrane having a MWCO 100-3500 Da or a membrane pore size between 0.5 and 2 nm. There can be an optional diafiltration, wherein a majority of the HMOs are retained in retentate thereby allowing at least water and preferably other small molecules to pass through the membrane. The small molecules can include monosaccharides, disaccharides, small bacterial metabolites, and salts.

Preferably, the NF step directly follows the centrifugation, that is the feed of the NF step is the supernatant containing the HMO of interest obtained after centrifugation. Optionally, the supernatant can be decolorized by using active charcoal (see below) before conducting the NF step. This nanofiltration step may advantageously be used to concentrate the supernatant and/or to remove ions, mainly monovalent ions, and organic materials having a molecular weight lower than that of the neutral HMO, such as monosaccharides and disaccharides. In one aspect of nanofiltration, the MWCO of the NF membrane is around 25-50% of the molecular weight of the HMO of interest, typically around 150-500 Da. In this regard, the neutral HMO of interest is accumulated in the NF retentate (NFR). The nanofiltration can be combined with diafiltration with water to remove, or to reduce the amount of, permeable salts such as monovalent ions more effectively. In a second aspect of nanofiltration, which is advantageously applicable for a supernatant that contains more lactose, the membrane has a MWCO of 600-3500 Da ensuring the retention of the tri- or higher neutral HMO and allowing at least a part of lactose to pass through the membrane, wherein the active (top) layer of the membrane is composed of polyamide, and wherein the MgSO₄ rejection factor on said membrane is around 20-90%, preferably 50-90% measured under sufficient cross-flow >0.3 m/s, TMP=7-10 bar at around 0.2 M concentration of MgSO₄ in water and a temperature around 20-25° C. The active or the top layer of the nanofiltration membrane suitable in this second aspect is preferably made of polyamide, yet preferably, the membrane is a thin-film composite (TFC) membrane. An example of suitable piperazine based polyamide TFC membranes is TriSep® UA60.

The NF step can be conducted under conditions used for conventional nanofiltration with tangential flow or cross-flow filtration with positive pressure compared to permeate side followed by diafiltration where both operations could be performed in a batch mode or preferably in multistage continuous mode. In a batch mode, the optional diafiltration is conducted by adding pure water to the retentate after the nanofiltration step disclosed above and continuing the filtration process with constant removal of permeate under the same or similar conditions as nanofiltration. The preferred mode of water addition is continuous, i.e. the addition flow rate is matching approximately the permeate flow rate. NF could be performed in a batch mode where retentate stream is recycled back to the feed tank and the diafiltration (DF) is done by adding purified or deionized water to the feed tank continuously. Most preferably, DF water is added after at least some pre-concentration by removing a certain amount of permeate. The higher concentration factor before the start of DF the better DF efficacy is achieved. After completion of DF, further concentration could be achieved by removing extra amount of permeate. Alternatively, NF could be performed in continuous mode preferably in a multi-loop system where a part of the retentate from each loop is transferred to the next loop. In this case, DF water could be added separately in each loop with the flow rate either matching the permeate flow rate in each loop or at a lower flow rate. Like in batch mode DF, to improve the efficacy of DF, less water or no water should be added in the e.g. first loop to achieve a higher concentration factor. The distribution of water in multi-loop system as well as other process parameters such as trans-membrane pressure, temperature and cross-flow is subject to routine optimization.

The convenient temperature range applied the NF step is between around 10 and around 80° C. Higher temperature provides a higher flux and thus accelerates the process. The membrane is expected to be more open for flow-through at higher temperatures, however this doesn't change the separation factors significantly. A preferred temperature range for conducting the nanofiltration separation according to the invention is around 15-45° C., such as 20-45° C.

A preferred applied pressure in the nanofiltration separation is about 2-50 bars, such as around 10-40 bars. Generally, the higher the pressure the higher the flux.

In certain embodiments, the method of the invention may comprise additional (one or more) NF steps, preferably following an active charcoal treatment (see below) and/or ion exchange treatment (see below) and/or chromatography on neutral solid phase (see below), wherein the main purpose is to concentrate the aqueous solution containing the neutral HMO of interest.

In some embodiments, the post-processing method can include polishing de-coloration of the clarified supernatant by active charcoal. In some iterations, an amount of charcoal relative to the HMOs is <100% by weight, preferably <10%. This can allow most of the HMOs to pass while retaining residual biomolecules, coloured compounds, and other hydrophobic molecules.

The optional active charcoal treatment may follow any of centrifugation, NF step or the ion exchange treatment step. The active charcoal treatment helps to remove colorizing agents and/or to reduce the amount of other water soluble contaminants, such as more hydrophobic or less polar metabolites, if required. Moreover, the active charcoal treatment removes residual or trace bio-macromolecules such as proteins, DNAs, RNAs or endotoxin that may remain incidentally after the previous steps.

A carbohydrate substance like an HMO of interest tends to bind to the surface of charcoal particles from its aqueous solution. Similarly, the colorizing agents are also capable to adsorb to the charcoal. While the carbohydrates and colour giving materials are adsorbed, water soluble materials that are not or are more weakly bound to the charcoal can be eluted with water. By changing the eluent from water to aqueous alcohol, e.g. methanol, ethanol, or iso-propanol, the adsorbed HMO can easily be eluted and collected in a separate fraction. The adsorbed colour giving substances would still remain adsorbed on the charcoal, thus both decolourization and partial desalination can be achieved simultaneously in this optional step. However, due to the presence of organic solvent (ethanol) in the elution solvent, the efficacy of decolorization is lower compared to the case when the elution is done with pure water. Under certain conditions, the HMO is not, or at least not substantially, adsorbed to the charcoal particles and elution with water gives rise to an aqueous solution of the HMO without a significant loss in its amount, while the colour giving substances remain adsorbed. In this case, there is no need to use organic solvent such as ethanol for elution. It is a matter of routine skills to determine the conditions under which the HMO would bind to the charcoal from its aqueous solution. For example, in one embodiment, a more diluted solution of the HMO or a higher amount of charcoal relative to the amount of the HMO is used, in another embodiment a more concentrated solution of the HMO and a lower amount of charcoal relative to the amount of the HMO is applied. The charcoal treatment can be conducted by adding charcoal powder to the aqueous solution of the HMO under stirring, filtering off the charcoal, re-suspending in aqueous ethanol under stirring and separating the charcoal by filtration. In higher scale purification, the aqueous solution of HMO is preferably loaded to a column packed with charcoal, which may optionally be mixed with celite, then the column is washed with the required eluent. The fractions containing the HMO are collected. Residual alcohol, if used for elution, may be removed from these fractions by e.g. evaporation, to give an aqueous solution of the HMO. Preferably, the active charcoal used is granulated. This ensures a convenient flow-rate without applying high pressure. Also preferably, the active charcoal treatment, more preferably the active charcoal chromatography is conducted at elevated temperature. At elevated temperature, the binding of colour bodies, residual proteins, etc. to the charcoal particles takes place in a shorter contact time, therefore the flow-rate can be conveniently raised. Moreover, the active charcoal treatment conducted at elevated temperature substantially reduces the total number of viable microorganisms (total microbial count) in the aqueous solution of the HMO, thus a sterile filtration step in the later phase of the method may not be necessary. The elevated temperature is at least 30-35° C., such as at least 40° C., at least 50° C., around 40-50° C. or around 60° C. Also preferably, the amount the applied charcoal is not more than around 10 weight % of the HMO contained in the load, more preferably around 2-6 weight %. This is economical, because all the benefits disclosed above can be conveniently achieved with a very low amount of charcoal. It is especially preferred when the active charcoal treatment, is conducted at elevated temperature with not more than around 10 weight %, preferably around 2-6 weight %, of active charcoal relative to the amount of the HMO of interest in the aqueous solution.

In some embodiments, the post-processing method can include concentration. For example, by one or more of evaporation, vacuum evaporation, nanofiltration, reverse osmosis, or forward osmosis.

In some embodiments, the post-processing method can include de-salting. For example, by passing the centrifuged supernatant or the post-treated centrifuged supernatant as disclosed above through a cation exchanger in H⁺-form, preferably a strongly acidic cation exchange resin. In this step, the positively charged materials can be removed from the feed solution as they bind to the resin with simultaneous release of protons from the exchanger. The solution of the HMO is contacted with the cation exchange resin in any suitable manner which would allow positively charged materials to be bound onto the cation exchange resin, and the HMO to pass through. This may further be followed by an acid adsorbing step. For example, passing it through or adding weakly basic resin. The resin is used preferably in a free base form. In this step, the protons generated in the previous step are captured by the resin's basic groups allowing to attract anions and thus to be removed from the feed solution as they bind to the protonated resin. The aqueous solution of the HMO is contacted with a weak basic anion exchange resin in any suitable manner which would allow the anions to be adsorbed onto the anion exchange resin, and the HMO to pass through. In overall, not only salts but colour bodies, biomolecules that contains ionizable groups (such as proteins, peptides, DNA and endotoxins), neutral or zwitter-ionic compounds containing ionizable functional groups (such as amino groups in metabolites including biogenic amines, amino acids) can be further removed by treatment with resin. In some embodiments, the de-salting is done through dialysis. In some embodiments, the de-salting is done through neutralization dialysis.

In some embodiments, the post-processing method can include sterile filtration of the HMOs.

In some embodiments, the post-processing method can include electrodialysis of the HMOs.

In some embodiments, the post-processing method can include performing chromatography separation/purification of neutral HMOs on a neutral solid phase. In this optional step, soluble hydrophobic impurities contained in the centrifuged supernatant or the post-treated centrifuged supernatant as disclosed above can be removed by subjecting this aqueous medium to a chromatography on a neutral solid phase, advantageously a reversed-phase chromatography on reversed-phase silicas and organic polymers, especially copolymers of styrene or divinylbenzene and methacrylate polymer. In one embodiment, the solid phase is a bromine functionalized PS-DVB hydrophobic stationary phase.

In some embodiments, the post-processing method can include solid HMO separation. For example, via crystallization. Alternatively, water removal can be used for solid HMO separation. For example, freeze-drying, spray-drying, drum-drying.

In some embodiments, the post-processing method comprises the following separation/purification steps in any order:
a) optional ultrafiltration (UF)
b) nanofiltration (NF), and
c) treatment with an ion exchange resin, and/or chromatography on a neutral solid phase.

In some embodiments, the post-processing method does not comprise electrodialysis.

Advantageously, step a) is conducted before step b). More advantageously, the step a) is conducted before any of the steps b) and c). Preferably, the method is performed in the order where step b) follows step a) and step c) follows step b).

In one embodiment, the post-processing method includes:
nanofiltration (NF) of the centrifuged supernatant and collecting the nanofiltration retentate (NFR),
treatment of the NFR with ion exchange resins and collecting the resin eluate (RE), and
chromatography of the RE.

In another embodiment, the post-processing method comprises:
nanofiltration (NF) of the centrifuged supernatant and collecting the nanofiltration retentate (NFR),
chromatography of the NFR and collecting the chromatography eluate (CE), and
treatment of the CE with ion exchange resins.

In some embodiments, the post-processing method may include an active charcoal treatment after centrifugation, NF, chromatography or ion exchange resin treatment.

In one embodiment, the post-processing method includes:
nanofiltration (NF) of the centrifuged supernatant and collecting the nanofiltration retentate (NFR),
activated charcoal treatment of the NFR and collecting the charcoal eluate (CCE), and
treatment of the CCE with ion exchange resins.

Preferably, the post-processing method includes:
nanofiltration (NF) of the centrifuged supernatant and collecting the nanofiltration retentate (NFR),
activated charcoal treatment of the NFR and collecting the charcoal eluate (CCE), and
treatment of the CCE with a strong cation exchange resin in H⁺-form and a weak base anion exchange resin in free base form.

More preferably, the post-processing method includes:
nanofiltration (NF) of the centrifuged supernatant and collecting the nanofiltration retentate (NFR),
activated charcoal treatment of the NFR and collecting the charcoal eluate (CCE), and
treatment of the CCE with a strong acid cation exchange resin in H⁺-form and a weak base anion exchange resin in free base form.

In some embodiments, the method may not include electrodialysis.

In another embodiment, the post-processing method includes:
nanofiltration (NF) of the centrifuged supernatant and collecting the nanofiltration retentate (NFR),
treatment of the NFR with ion exchange resins and collecting the resin eluate (RE), and
activated charcoal treatment of the RE.

Preferably, the post-processing method includes:
nanofiltration (NF) of the centrifuged supernatant and collecting the nanofiltration retentate (NFR),
treatment of the NFR with a strong acid cation exchange resin in H⁺-form and a weak base anion exchange resin in free base form, and collecting the resin eluate (RE), and
activated charcoal treatment of the RE.

More preferably, the post-processing method includes:
nanofiltration (NF) of the centrifuged supernatant and collecting the nanofiltration retentate (NFR),
treatment of the NFR with a strong acid cation exchange resin in H⁺-form and a weak base anion exchange resin in free base form, and collecting the resin eluate (RE), and
activated charcoal treatment of the RE.

In some iterations, the post-processing method does not include electrodialysis.

Yet in another embodiment, the method includes:
nanofiltration (NF) of the centrifuged supernatant and collecting the nanofiltration retentate (NFR),
chromatography of the NFR and collecting the chromatography eluate (CE), and
activated charcoal treatment of the CE.

Yet in another embodiment, the post-processing method includes:
nanofiltration (NF) of the centrifuged supernatant and collecting the nanofiltration retentate (NFR),
activated charcoal treatment of the NFR and collecting the charcoal eluate (CCE), and
chromatography of the CCE.

Yet in another embodiment, the post-processing method includes:
nanofiltration (NF) of the centrifuged supernatant and collecting the nanofiltration retentate (NFR),
chromatography of the NFR and collecting the chromatography eluate (CE),
activated charcoal treatment of the CE and collecting the charcoal eluate (CCE), and
treatment of the CCE with ion exchange resins.

Yet in another embodiment, the post-processing method includes:
nanofiltration (NF) of the centrifuged supernatant and collecting the nanofiltration retentate (NFR),
chromatography of the NFR and collecting the chromatography eluate (CE),
treatment of the CE with ion exchange resins and collecting the resin eluate (RE), and
activated charcoal treatment of the RE.

Yet in another embodiment, the post-processing method includes:
nanofiltration (NF) of the centrifuged supernatant and collecting the nanofiltration retentate (NFR),
treatment of the NFR with ion exchange resins and collecting the resin eluate (RE),
chromatography of the RE and collecting the chromatography eluate (CE), and
activated charcoal treatment of the CE.

Yet in another embodiment, the post-processing method includes:
nanofiltration (NF) of the centrifuged supernatant and collecting the nanofiltration retentate (NFR),
treatment of the NFR with ion exchange resins and collecting the resin eluate (RE)
activated charcoal treatment of the RE and collecting the charcoal eluate (CCE), and
chromatography of the CCE.

Yet in another embodiment, the post-processing method includes:
nanofiltration (NF) of the centrifuged supernatant and collecting the nanofiltration retentate (NFR),
activated charcoal treatment of the NFR and collecting the charcoal eluate (CCE),
treatment of the CCE with ion exchange resins and collecting the resin eluate (RE), and
chromatography of the RE.

Yet in another embodiment, the post-processing method includes:
nanofiltration (NF) of the centrifuged supernatant and collecting the nanofiltration retentate (NFR),
activated charcoal treatment of the NFR and collecting the charcoal eluate (CCE),
chromatography of the CCE and collecting the chromatography eluate (CE), and
treatment of the CE with ion exchange resins.

In some embodiments, the clarified supernatant is purified via the optional ultrafiltration or microfiltration, the nanofiltration, the de-salting, and the polishing, followed by the concentration and the solid HMO product separation.

### EXAMPLES

The following examples provide some data results for embodiments of the disclosure, but should not be considered limiting. Carbohydrate and impurity content were quantified by calibrated HPLC and/or HPAEC. Soluble proteins were quantified by Bradford assay. To quantify residual total suspended solids, Absorbance at 600 nm (Abs600) was measured with Thermo Fisher spectrophotometer type 1510 in disposable PMMA 1.5 ml cuvettes with 1 cm path length relative to pure water absorbance which was set to 0. In most cases Abs600 values were then corrected by the Abs600 value obtained for the micro-filtered sample by subtraction and defined here as OD600 values. Depending on the scale, centrifugation was performed in three different lab-scale centrifuges as indicated in examples in Eppendorf 2 ml microcentrifuge tubes or in PP falcon tubes of 15 ml or 50 ml. The larger lab scale centrifugation was performed in 1 l vessels.

Reported RCF or “g-force” values are max RCF calculated for the max radius of rotation according to rotor specifications. However, because of different centrifuge rotor geometry sample tube geometry and volume difference, spinning results especially Abs600 values obtained in different centrifuge rotors and with different volumes under the same max RCF and exposure time, might be different due to different g-force gradient within the bulk of the sample. Therefore, the same set of experiments were performed under exact same conditions, i.e. in the same centrifuge, in the same size tubes and similar volumes.

Results clearly indicates that heat treatment along can accelerate the settling velocities by up to 9-fold at this particular pH value (4.1). Further dilution and heat treatment improve substantially the relative rates by up to 5000-fold (best at 10× dilution) and relative throughput by up to 500-fold. In addition, residual suspended solids are better minimized after pre-heating at higher temperature. As an example, OD600 of supernatants obtained for undiluted broth at RCF=3000 g for 3 min could be substantially reduced by >5-fold from 1.94 to 0.31 after pre-heating of the broth at 90° C. or by 70-fold to as low as 0.024 after 3× dilution and heat treatment under the same centrifugation conditions.

### Example 1

### Soluble Protein Reduction in 2-′FL Broth Supernatant by pH Adjustment and Heat-Treatment

2′-FL-containing broth was generated by fermentation using a genetically modified E. coli strain of LacZ⁻, LacY⁺ phenotype, wherein said strain comprises a recombinant gene encoding an α-1,2-fucosyltransferase enzyme which is able to transfer fucose of GDP-fucose to the internalized lactose and genes encoding a biosynthetic pathway to GDP-fucose. The fermentation was performed by culturing the strain in the presence of exogenously added lactose and a suitable carbon source, thereby producing 2′-FL which was accompanied with DFL and unreacted lactose as major carbohydrate impurities in the fermentation broth.

Several samples of 2′-FL fermentation broth were subjected to pH adjustment by dropwise addition of aqueous 25% H₂SO₄ with stirring at room temperature, pH was measured after equilibration for 20 min and after 2 h. Samples of the obtained liquids (2×2 ml each) were centrifuged at room temperature (T=+22° C.) at RCF=13000 g (11600 RPM) for 3 min in Thermo Scientific Heraeus Pico17 centrifuge. Other samples were thermostated at 60° C. for 10 min, cooled down to room temperature and centrifuged under the same conditions. Obtained clarified supernatants were micro-filtered and analyzed by Bradford test for soluble protein content. Results are summarized in the table below. Results clearly indicate that a combination of low pH (<4) and a moderate heat treatment (60° C.) can reduce soluble proteins up to 10-fold relative to supernatant of the untreated broth while treatment alone by either heat or pH-adjustment at room temperature is not so efficient. The above conditions (60° C. for 10 min and pH) are given as examples and not as limiting values. The optimal conditions (pH, temperature and exposure time) are subject to further routine optimization that could be done by any skilled person.

| Proteins [mg/l] in fermentation broth | | | |
|---|---|---|---|
| supernatant vs pH at r.t. and after 60° C./10 min | | | |
| pH after adjustment | Proteins [mg/l] | Proteins [mg/l] | pH after |
| and equilibration | in supernatant | in supernatant after | 2 h at |
| for 20 min | at r.t. | 60° C./10 min | r.t. |
| 6.3 | 3561 | 3719 | 6.3 |
| (no adjustment) | | | |
| 5.0 | 2777 | 2265 | 5.12 |
| 4.5 | 2213 | 1320 | 4.64 |
| 4.2 | 1839 | 1135 | 4.34 |
| 3.9 | 1574 | 808 | 4.14 |
| 3.6 | 1248 | 1012 | 3.96 |
| 3.3 | 900 | 390 | 3.71 |

### Example 2

### Soluble Protein Reduction and Yield in LNFP-I Fermentation Broth Supernatant After pH Adjustment, Dilution and Heat-Treatment

LNFP-I-containing broth was generated by fermentation using a genetically modified E. coli strain of LacZ⁻, LacY⁺ phenotype, wherein said strain comprises a recombinant gene encoding β-1,3-N-acetyl-glucosaminyl transferase which is able to transfer the GlcNAc of UDP-GlcNAc to the internalized lactose, a recombinant gene encoding a β-1,3-galactosyl transferase which is able to transfer the galactosyl residue of UDP-Gal to the N-acetyl-glucosaminylated lactose (lacto-N-triose II or LNT-2) forming LNT (lacto-N-tetraose), alpha-1,2-fucosyltransferase enzyme which is able to transfer fucose of GDP-fucose to LNT and genes encoding a biosynthetic pathway to UDP-GIcNAc, UDP-Gal, and GDP-fucose. The fermentation was performed by culturing the strain in the presence of exogenously added lactose and a suitable carbon source, thereby producing LNFP-I which was accompanied with some 2′-FL, LNT and unreacted lactose as major carbohydrate impurities in the fermentation broth.

In the end of fermentation, 25% H₂SO₄ was added slowly to adjust pH to 4.0. Samples of the obtained broth was subjected to dilution (1×=no dilution as a reference, 2× and 3×) to ca 50 ml total in 50 ml NerbePlus centrifuge PP tubes and spinned at room temperature (T=+22° C.) in Beckman Coulter Allegra X-30R centrifuge equipped with C0650 Fixed-Angle Conical Tube Rotor at RCF=10000 g (9851 RPM) for 10 min. The same set of samples was pre-treated by incubation in water bath at T=+60° C. with periodic shaking and cooled down to room temperature before centrifugation. The supernatants were carefully removed from the pellet and analyzed. Samples of obtained pellets (0.4 g) were diluted with water by 10× to total 4.0 g followed by short heating to +95° C. for 5 min, centrifuged at RCF=13000 g (11600 RPM) for 3 min in Thermo Scientific Heraeus Pico17 centrifuge and obtained supernatants were analyzed by HPLC and for protein content by Bradford test. Also, absorbance at 600 nm was measurement relative to pure water as an indication of suspended solid content.

The data are summarized in the table below. The results clearly indicate protein reduction in the supernatants after heat treatment by ca 4-fold in agreement with example #1 at pH=4 and an improvement in the yield of LNFP-I after both dilution and heat treatment, i.e. from 49.5% in the supernatant of the untreated broth to 76.8% from 3×-diluted broth and to 90.8% after both 3×-dilution and heat treatment. In addition, total suspended solids are better reduced after dilution and heat-treatment, which is apparent by visual evaluation (i.e. cloudy supernatants were 10 obtained without dilution) and quantification by absorbance measurement at 600 nm.

| | | | After | | BWM | | | | | | | |
|---|---|---|---|---|---|---|---|---|---|---|---|---|
| Sample | | Amount | dilution | Pellet | (BWM/ | | Proteins | Abs600 | LNFP-I, | LNFP-I | 2′-FL, | 2′-FL, |
| # | Description | (g) | (g) | (g) | dilution) | pH | mg/l | nm | g/l | yield | g/l | yield |
| 1 | Broth Supernatant | 52.3 | Same | 24.059 | 45.9% | 4.1 | 2478 | 1.813 | 37.48 | 49% | 29.57 | 69% |
| | 10000 g/10 min | | | | (45.9%) | | | | | | | |
| 2 | Pellet/10x/95° C.- | 0.4217 | 4.221 | | | | | | 4.27 | | 1.50 | |
| | 5 min/13000 g-3 min | | | | | | | | | | | |
| 3 | Broth 2x | 26.585 | 53.286 | 11.086 | 20.8% | 4.1 | 1062 | 0.692 | 16.89 | 69% | 12.17 | 81% |
| | Supernatant | | | | (41.7%) | | | | | | | |
| | 10000 g/10 min | | | | | | | | | | | |
| 4 | Pellet/10x/95° C.- | 0.4257 | 4.262 | | | | | | 2.77 | | 1.09 | |
| | 5 min/13000 g-3 min | | | | | | | | | | | |
| 5 | Broth 3x | 17.039 | 51.138 | 6.701 | 13.1% | 4.2 | 659 | 0.360 | 11.55 | 77% | 8.12 | 90% |
| | Supernatant | | | | (39.3%) | | | | | | | |
| | 10000 g/10 min | | | | | | | | | | | |
| 6 | Pellet/10x/95° C.- | 0.4068 | 4.065 | | | | | | 2.28 | | 0.56 | |
| | 5 min/13000 g-3 min | | | | | | | | | | | |
| 7 | Broth/60° C.-1 h | 51.404 | same | 20.817 | 40.5% | 4.38 | 623 | 0.767 | 46.74 | 66% | 29.31 | 65% |
| | Supernatant | | | | (40.5%) | | | | | | | |
| | 10000 g/10 min | | | | | | | | | | | |
| 8 | Pellet/10x/95° C.- | 0.3934 | 3.930 | | | | | | 3.37 | | 2.17 | |
| | 5 min/13000 g-3 min | | | | | | | | | | | |
| 9 | Broth 2x/60° C.-1 h | 25.174 | 50.353 | 9.334 | 18.5% | 4.3 | 290 | 0.242 | 20.88 | 86% | 8.66 | 85% |
| | Supernatant | | | | (37.1%) | | | | | | | |
| | 10000 g/10 min | | | | | | | | | | | |
| 10 | Pellet/10x/95° C.- | 0.4305 | 4.306 | | | | | | 1.49 | | 0.66 | |
| | 5 min/13000 g-3 min | | | | | | | | | | | |
| 11 | Broth 3x/60° C.-1 h | 17.033 | 51.196 | 6.051 | 11.8% | 4.3 | 203 | 0.122 | 13.22 | 91% | 8.25 | 93% |
| | Supernatant | | | | (35.5%) | | | | | | | |
| | 10000 g/10 min | | | | | | | | | | | |
| 12 | Pellet/10x/95° C.- | 0.4363 | 4.362 | | | | | | 0.98 | | 0.46 | |
| | 5 min/13000 g-3 min | | | | | | | | | | | |
| 13 | Broth 10x/95° C.- | 0.4320 | 4.323 | | | | 27 | 0.049 | 4.15 | | 1.66 | |
| | 5 min/13000 g-3 min | | | | | | | | | | | |

Amount is the total amount of broth used in a sample before pre-treatment; in case of a pellet, the amount is a sample amount taken from pellet after centrifugation BWM (bio-wet mass) is the ratio between mass of pellet after decanting the supernatant divided by the initial mass of diluted broth. (In parenthesis the normalized BWM was calculated as well where mass of the broth was used before dilultion).

### Example 3

### Settling Velocities vs Dilution and Pre-Heat

The same LNFP-I broth from example 2 (pH=4.0) was used to prepare samples with various dilution factors (1×=no dilution as a reference, 2×, 3×, 5×, and 10×) by adding relevant amount of water. For example, for 3× dilution 5.00 g of broth was diluted to 15.00 g total mass with 10.00 g of water. 15 ml of each obtained sample in 15 ml NerbePlus centrifuge PP falcon tubes was pre-heated to 60° C., 70° C., 80° C. and 90° C. for various duration from 5 min to 1 h in water bath with periodic gentle shaking to avoid foaming as summarized in the tables below. After pre-heat, samples were cooled immediately to room temperature in cold water bath. After pre-treatment, all samples were gently re-suspended at the same time by slow turning upside down few times to avoid foaming and allowed to stand vertically at ambient temperature. The height of clear supernatant layer was periodically measured in mm vs time (raw data are not included in the table). The initial settling velocities were obtained from linear regression of height vs time with the forced line intercept at 0 of the several initial points and summarized in the table. The relative sedimentation rates were calculated as a ratio of the obtained velocities vs reference settling velocity for the untreated broth. The relative broth throughput was calculated as the relative rate divided by dilution factor, so the rate was normalized per actual broth amount before dilution. Results clearly indicates that heat treatment alone can accelerate the settling velocities by up to 9-fold at this particular pH value (4.1). Further dilution and heat treatment improve substantially the relative rates by up to 5000-fold (best at 10× dilution) and relative throughput by up to 500-fold. In addition, residual suspended solids are better minimized after pre-heating at higher temperature. As an example, OD600 of supernatants obtained for undiluted broth at RCF=3000 g for 3 min could be substantially reduced by >5-fold from 1.94 to 0.31 after pre-heating of the broth at 90° C. or by 70-fold to as low as 0.024 after 3× dilution and heat treatment under the same centrifugation conditions.

| | | Sed. | | | Relative | Abs_600 | Abs_600 | Abs_600 | Abs_600 |
|---|---|---|---|---|---|---|---|---|---|
| Pre-treatment | | rate, | | Relative | through- | RCF3000/ | RCF10000/ | after MF | before |
| (No dilution) | test # | mm/h | R² | rate | put | 3 min | 10 min | (0.22 μm) | centrifuge |
| No (reference) | 1 | 0.0488 | 0.887 | 1 | 1 | 2.153 | 0.482 | 0.2168 | >>10 |
| 60° C./10 min | 2 | 0.0446 | 0.9242 | 0.91 | 0.91 | 1.894 | 0.348 | | |
| 60° C./20 min | 3 | 0.0897 | 0.9119 | 1.84 | 1.84 | 1.872 | 0.354 | | |
| 60° C./40 min | 4 | 0.0243 | 0.9069 | 0.50 | 0.50 | 1.705 | 0.352 | 0.233 | |
| 60° C./60 min | 5 | 0.0574 | 0.8495 | 1.18 | 1.18 | 2.187 | 0.344 | | |
| 70° C./5 min | 6 | 0.0282 | 0.9026 | 0.58 | 0.58 | 2.082 | 0.336 | | |
| 70° C./10 min | 7 | 0.1645 | 0.8773 | 3.37 | 3.37 | 2.068 | 0.374 | | |
| 70° C./20 min | 8 | 0.0556 | 0.9691 | 1.14 | 1.14 | 2.292 | 0.343 | 0.169 | |
| 70° C./40 min | 9 | 0.044 | 0.9367 | 0.90 | 0.90 | 2.21 | 0.329 | | |
| 80° C./5 min | 10 | 0.4037 | 0.9813 | 8.27 | 8.27 | 2.365 | 0.347 | | |
| 80° C./10 min | 11 | 0.0898 | 0.9121 | 1.84 | 1.84 | 1.999 | 0.305 | | |
| 80° C./20 min | 12 | 0.4534 | 0.9817 | 9.29 | 9.29 | 2.124 | 0.295 | 0.162 | |
| 80° C./40 min | 13 | 0.3019 | 0.964 | 6.19 | 6.19 | 1.483 | 0.278 | | |
| 90° C./5 min | 14 | 0.2055 | 0.9924 | 4.21 | 4.21 | 1.826 | 0.315 | | |
| 90° C./10 min | 15 | 0.1994 | 0.9525 | 4.09 | 4.09 | 1.183 | 0.266 | | |
| 90° C./20 min | 16 | 0.0474 | 0.9227 | 0.97 | 0.97 | 0.592 | 0.258 | 0.148 | |
| 90° C./40 min | 17 | 0.006 | 0.7617 | 0.12 | 0.12 | 0.454 | 0.248 | | |

| Pre- | | | | | | | | | |
|---|---|---|---|---|---|---|---|---|---|
| treatment | | | | | | | | | |
| (all samples | | Sed. | | | Relative | Abs_600 | Abs_600 | Abs_600 | Abs_600 |
| with 2x | test | rate | | Relative | through- | RCF3000/ | RCF10000/ | after MF | Before |
| dilution) | # | (mm/h) | R² | rate | put | 3 min | 10 min | (0.22 μm) | centrifuge |
| r.t. | 18 | 1.55 | 0.879 | 31.76 | 15.9 | 0.4741 | 0.1315 | 0.0811 | >4.0 |
| 60° C./10 min | 19 | 1.1 | 0.9111 | 22.54 | 11.3 | 0.4462 | 0.1297 | | |
| 60° C./20 min | 20 | 0.8893 | 0.9876 | 18.22 | 9.1 | 0.4513 | 0.1426 | | |
| 60° C./40 min | 21 | 1.1 | 0.9111 | 22.54 | 11.3 | 0.366 | 0.1399 | 0.0867 | |
| 60° C./60 min | 22 | 1.25 | 0.898 | 25.61 | 12.8 | 0.4208 | 0.1327 | | |
| 70° C./5 min | 23 | 1.1 | 0.9111 | 22.54 | 11.3 | 0.3915 | 0.1339 | | |
| 70° C./10 min | 24 | 1.26 | 0.9163 | 25.82 | 12.9 | 0.406 | 0.1365 | | |
| 70° C./20 min | 25 | 1.275 | 0.942 | 26.13 | 13.1 | 0.3786 | 0.1266 | 0.0768 | |
| 70° C./40 min | 26 | 0.8607 | 0.9995 | 17.64 | 8.8 | 0.2791 | 0.1656 | | |
| 80° C./5 min | 27 | 0.975 | 0.9778 | 19.98 | 10.0 | 0.3995 | 0.128 | | |
| 80° C./10 min | 28 | 0.7729 | 0.9983 | 15.84 | 7.9 | 0.3061 | 0.1244 | | |
| 80° C./20 min | 29 | 0.7672 | 0.9955 | 15.72 | 7.9 | 0.271 | 0.1073 | 0.063 | |
| 80° C./40 min | 30 | 0.7615 | 0.9895 | 15.60 | 7.8 | 0.2208 | 0.1027 | | |
| 90° C./5 min | 31 | 0.7615 | 0.9895 | 15.60 | 7.8 | 0.2465 | 0.105 | | |
| 90° C./10 min | 32 | 0.7805 | 0.9916 | 15.99 | 8.0 | 0.1843 | 0.1134 | | |
| 90° C./20 min | 33 | 0.9599 | 0.9979 | 19.67 | 9.8 | 0.1341 | 0.1112 | 0.0579 | |
| 90° C./40 min | 34 | 1.025 | 0.9633 | 21.00 | 10.5 | 0.126 | 0.0859 | | |

| | | Initial | | | | | | | |
|---|---|---|---|---|---|---|---|---|---|
| Pre-treatment | | sed. | | | Rel. | Abs_600 | Abs_600 | Abs_600 | Abs_600 |
| (Dilution 3x, | test | rate, | | Rel. | through- | RCF3000/ | RCF10000/ | after MF | before |
| 5x, and 10x) | # | mm/h | R² | rate | put | 3 min | 10 min | (0.22 μm) | centrifuge |
| 3x/r.t. | 35 | 7.77 | 0.9185 | 159.2 | 53.1 | 0.1986 | 0.0829 | 0.0478 | 3.954 |
| 3x/60° C./10 min | 36 | 8.82 | 0.9024 | 180.7 | 60.2 | 0.1518 | 0.072 | | |
| 3x/60° C./20 min | 37 | 9.26 | 0.9019 | 189.9 | 63.3 | 0.2324 | 0.0706 | 0.0434 | |
| 3x/60° C./40 min | 38 | 8.17 | 0.9018 | 167.4 | 55.8 | 0.1487 | 0.0748 | | |
| 3x/60° C./60 min | 39 | 8.14 | 0.908 | 166.7 | 55.6 | 0.1413 | 0.0738 | | |
| 3x/70° C./5 min | 40 | 8.51 | 0.8714 | 174.3 | 58.1 | 0.1656 | 0.07 | | |
| 3x/70° C./10 min | 41 | 9.83 | 0.9047 | 201.4 | 67.1 | 0.1875 | 0.0891 | | |
| 3x/70° C./20 min | 42 | 8.17 | 0.8813 | 167.4 | 55.8 | 0.1354 | 0.0703 | 0.0395 | |
| 3x/70° C./40 min | 43 | 8.84 | 0.9084 | 181.2 | 60.4 | 0.1282 | 0.0656 | | |
| 3x/80° C./5 min | 44 | 10.45 | 0.9275 | 214.1 | 71.4 | 0.189 | 0.0673 | | |
| 3x/80° C./10 min | 45 | 9.42 | 0.9585 | 193.1 | 64.4 | 0.1203 | 0.0633 | | |
| 3x/80° C./20 min | 46 | 5.68 | 0.9642 | 116.4 | 38.8 | 0.1239 | 0.0658 | 0.0399 | |
| 3x/80° C./40 min | 47 | 74.4 | −143.4 | 1524.6 | 508.2 | 0.116 | 0.0881 | | |
| 3x/90° C./5 min | 48 | 5.78 | 0.9885 | 118.4 | 39.5 | 0.0948 | 0.0571 | | |
| 3x/90° C./10 min | 49 | 7.16 | 0.9943 | 146.7 | 48.9 | 0.0853 | 0.0573 | | |
| 3x/90° C./20 min | 50 | 6.73 | 0.9938 | 137.9 | 46.0 | 0.0998 | 0.0553 | 0.0384 | |
| 3x/90° C./40 min | 51 | 43.2 | −1.178 | 885.2 | 295.1 | 0.0629 | 0.0504 | | |
| 5x/r.t. | 52 | 21.43 | 0.8608 | 439.1 | 87.8 | 0.0622 | 0.0394 | 0.0249 | 3.607 |
| 5x/60° C./20 min | 53 | 20 | 0.8783 | 409.8 | 82.0 | 0.1327 | 0.0429 | 0.022 | |
| 5x/70° C./10 min | 54 | 22.43 | 0.9462 | 459.6 | 91.9 | 0.06 | 0.0354 | 0.0226 | |
| 5x/80° C./10 min | 55 | 26.57 | 0.9812 | 544.5 | 108.9 | 0.0523 | 0.0325 | 0.0202 | |
| 5x/90° C./10 min | 56 | 45.14 | 0.9563 | 925.1 | 185.0 | 0.0667 | 0.0302 | 0.019 | |
| 10x/r.t. | 57 | 81 | N/A | 1659.8 | 166.0 | 0.0309 | 0.0231 | 0.0095 | 3.0666 |
| 10x/60° C./20 min | 58 | 45.71 | 0.9725 | 936.8 | 93.7 | 0.0457 | 0.0198 | 0.009 | |
| 10x/70° C./10 min | 59 | 73.2 | 0.6938 | 1500.0 | 150.0 | 0.0536 | 0.0167 | 0.01 | |
| 10x/80° C./10 min | 60 | 99.6 | 0.9984 | 2041.0 | 204.1 | 0.0312 | 0.0406 | 0.0085 | |
| 10x/90° C./10 min | 61 | 270 | N/A | 5532.8 | 553.3 | 0.0254 | 0.0164 | 0.0085 | |

### Example 4

### Settling Velocities vs pH, Dilution and Pre-Heat

LNFP-I-containing broth was generated on 2 l scale as described in example 2 (Feed #0), but without pH adjustment (pH=6.5). Part of the obtained broth (500.00 g) was diluted 3× times to total of 1500.00 g by addition of 1000.00 g of DI water in 2 l glass bottle. Then in was placed in water bath at 80° C. with periodic manual shaking to reach T=+70° C. inside in 50 min then placed immediately to cold water bath (Feed #1). To another 500.00 g portion of the broth 20 ml of 25% H₂SO₄ was added dropwise with stirring and diluted to total of 1500.00 g with DI water (pH 2.95) and heated to 70° C. in 30 min, then cooled down as above (Feed #2). Feed #2 had a lower viscosity as it was apparent from much better stirrability and flowability. In addition, from simple visual inspection, Feed #2 contained larger particles, i.e. with d>1 mm, which was not the case with Feed #1 (pH=6.5) and original undiluted Feed #0. Thus, an apparent self-flocculation occurred after pH-adjustment and heat-treatment without addition of flocculation agents. 15 ml samples in 15 ml PP centrifuge falcon tubes (total liquid h₀=105 mm, ID=15 mm) of each feed #0, #1, or #2 were allowed to stand vertically at ambient T=+22° C. with periodic measurement of clear supernatant layer height. Also, Feed #1 and #2 (V=ca 1.5 l in 2 l glass bottles, h₀=112 mm, ID=125 mm) were allowed to settle as well but at T=+5° C. The results in the table below clearly indicate substantial influence of pH on sedimentation velocities, i.e. by 40-fold in this example after dilution and pre-heat. Moreover, the combination of pH-adjustment, 3× dilution and heat treatment improves the settling rate by ca. 22000 fold or relative throughput by 7000-fold compared to untreated broth.

Settling distances in mm vs time and estimated settling velocities for untreated
broth (Feed #0), 3x-diluted and heat-treated to 70° C. (Feed #1, pH =
6.5) and pH-adjusted, diluted and heat-treated broth (Feed #2, pH = 3.0)

| | | Feed #0 in | Feed #1 in | Feed #2 in | Feed #1 in | Feed #2 in |
|---|---|---|---|---|---|---|
| time, | | 15 ml tube/ | 15 ml tube/ | 15 ml tube/ | 2 l bottle/ | 2 l bottle/ |
| min | | T = 22° C. | T = 22° C. | T = 22° C. | T = +5° C. | T = +5° C. |
| 0 | | 0 | 0 | 0 | 0 | 0 |
| 8 | | 0 | 0 | 6 | | |
| 12 | | 0 | 0 | 10 | | |
| 16 | | 0 | 0 | 14 | | |
| 22 | | 0 | 0 | 19 | | |
| 30 | | 0 | 0 | 27 | | |
| 37 | | 0 | | | 0 | 14 |
| 40 | | 0 | 0.5 | 32 | | |
| 45 | | 0 | | | 0 | 17 |
| 60 | | 0 | 1 | 37 | 0 | 22 |
| 90 | | 0 | 2 | 40 | 1 | 33 |
| 120 | | 0 | 2.5 | 42 | 2 | 43 |
| 180 | | 0 | 4 (not clear) | 44 | 4 | 50 |
| 300 | | 0 | 6 (not clear) | 45 | 7 (not clear) | 54 |
| 420 | | 0 | | 46 | 8 (not clear) | 56 |
| 1440 | (24 h) | 0 | 39 (not clear) | 47 | 56 (not clear) | 64 |
| 7200 | (5 days) | 0.2 | | | | |
| 21600 | (15 days) | 1 | | | | |

| | | | | | |
|---|---|---|---|---|---|
| 26 days | 3.5 | | | | |
| Initial settling | 4.00 · 10⁻⁵ | 0.021 | 0.877 | 0.0126 | 0.369 |
| velocity, mm/min | | | | | |
| Initial settling | 0.00240 | 1.28 | 52.60 | 0.76 | 22.14 |
| velocity, mm/h | | | | | |
| R² | 0.95 | 0.97 | 0.993 | 0.59 | 0.9984 |
| Relative rate | 1 | 533 | 21915 | 315 | 9225 |
| Relative | 1 | 178 | 7305 | 105 | 3075 |
| throughput | | | | | |

Values in bold are used for linear regression with the forced line intercept at (t = 0, h = 0).

### Example 5

### Centrifugation at Various RCF and Time and Two pH Values.

Feed #1 (pH=6.5/3×/70° C.) and Feed #2 (pH=2.95/3×/70° C.) from the previous example 4 were distributes in 2 ml Eppendorf microcentrifuge tubes and rotated in Thermo Scientific Heraeus Pico17 centrifuge at various RCF and duration. Supernatants were carefully removed and analysed by Abs600 measurements, remaining pellets were weighted and BWM calculated. For each test condition 2 samples were centrifuged, and supernatants combined. BWM values are reported as an average from two samples. The results in the tables below clearly indicate a much better clarification at pH=3.0 compared to pH=6.5, i.e. the clarification efficacy is approximately 50× better as pre-treated broth at pH=3.0 could be sedimented even at RCF as low as 200 g providing comparable Abs600 values with the supernatants obtained at 10000 g at pH=6.5. Furthermore, RCF=10000 g for 8 min is not enough to reduce OD600 below 0.1 at pH=6.5 in case of Feed #1. However, Feed #2 (pH=3.0) could be efficiently clarified to OD600<0.1 at RCF=3000 g for just 1 min.

Centrifugation of the 3x-diluted and heat-treated broth at pH = 6.5 (Feed #1)

| | | | | | | Abs | Abs_600 | |
|---|---|---|---|---|---|---|---|---|
| | Sample | | BWM | RCF | time, | 600 nm, | after MF, | |
| Description | # | Pellet stability | average | (g) | min | Supernatant | base-line | OD600 |
| Supernatant | 1 | not stable, | 45.45% | 3000 | 1 | 2.618 | 0.271 | 2.347 |
| @3000 g/1 min | | supernatant was | | | | | | |
| | | not fully removed | | | | | | |
| Supernatant | 2 | not stable, | 37.18% | 3000 | 2 | 1.984 | | 1.713 |
| @3000 g/2 min | | supernatant was | | | | | | |
| | | not fully removed | | | | | | |
| Supernatant | 3 | more stable, | 27.59% | 3000 | 4 | 1.235 | | 0.964 |
| @3000 g/4 min | | most supernatant | | | | | | |
| | | removed | | | | | | |
| Supernatant | 4 | more stable, | 23.74% | 3000 | 8 | 0.74 | | 0.469 |
| @3000 g/8 min | | most supernatant | | | | | | |
| | | removed | | | | | | |
| Supernatant | 5 | not stable, | 34.45% | 5000 | 1 | 2.495 | | 2.224 |
| @5000 g/1 min | | supernatant was | | | | | | |
| | | not fully removed | | | | | | |
| Supernatant | 6 | not stable, | 26.36% | 5000 | 2 | 1.396 | | 1.125 |
| @5000 g/2 min | | supernatant was | | | | | | |
| | | not fully removed | | | | | | |
| Supernatant | 7 | stable | 23.27% | 5000 | 4 | 0.9 | | 0.629 |
| @5000 g/4 min | | | | | | | | |
| Supernatant | 8 | stable | 20.31% | 5000 | 8 | 0.579 | | 0.308 |
| @5000 g/8 min | | | | | | | | |
| Supernatant | 9 | “liquid”, but | 24.65% | 10000 | 1 | 1.84 | | 1.569 |
| @10000 g/1 min | | most supernatant | | | | | | |
| | | removed | | | | | | |
| Supernatant | 10 | stable | 21.98% | 10000 | 2 | 0.905 | | 0.634 |
| @10000 g/2 min | | | | | | | | |
| Supernatant | 11 | stable | 19.43% | 10000 | 4 | 0.612 | | 0.341 |
| @10000 g/4 min | | | | | | | | |
| Supernatant | 12 | stable | 17.63% | 10000 | 8 | 0.485 | | 0.214 |
| @10000 g/8 min | | | | | | | | |
| Supernatant | 13 | stable | 12.14% | 17000 | 40 | 0.298 | | 0.027 |
| @17000 g/40 min | | | | | | | | |
| 3 kDa | 14 | | | | | 0.118 | | −0.153 |
| centrifugal | | | | | | | | |
| UF permeate | | | | | | | | |
| from Feed #1 | | | | | | | | |
| MF of #13 | 15 | | | | | 0.271 | 0.271 | 0 |
| 0.22 um Nylon filter | | | | | | | | |
| (base line for | | | | | | | | |
| samples #2-13) | | | | | | | | |
| Feed #1 as is | 16 | | | | | 3.933 | | N/A |
| | | | | | | (dilution | | |
| | | | | | | required) | | |
| Feed #0 as is | 17 | | | | | 3.81 | | N/A |
| | | | | | | (dilution | | |
| | | | | | | required) | | |
| Feed #0 | 18 | | 49.74% | 10000 | 90 | 0.67 | 0.444 | 0.226 |
| RCF = 10000 g/ | | | | | | | | |
| 3 × 30 min | | | | | | | | |
| MF of Sample | 19 | | | | | 0.444 | | 0 |
| #18 via RC | | | | | | | | |
| 0.45 μm filter | | | | | | | | |

| | | | | | | | Abs_600 | |
|---|---|---|---|---|---|---|---|---|
| | Sample | Pellet | BWM | RCF | time, | Abs_600 nm | after MF | |
| Description | # | stability | average | (g) | min | Supernatant | (base-line) | OD600 |
| RCF = 1500 g/1 | 1 | stable | 22.13% | 1500 | 1 | 0.211 | 0.0357 | 0.1753 |
| min | | | | | | | | |
| RCF = 1500 g/2 | 2 | stable | 23.32% | 1500 | 2 | 0.164 | | 0.1283 |
| mir | | | | | | | | |
| RCF = 1500 g/4 | 3 | stable | 18.55% | 1500 | 4 | 0.126 | | 0.0903 |
| min | | | | | | | | |
| RCF = 1500 g/8 | 4 | stable | 20.83% | 1500 | 8 | 0.142 | | 0.1063 |
| min | | | | | | | | |
| RCF = 500 g/1 | 5 | stable | 27.94% | 500 | 1 | 0.389 | | 0.3533 |
| min | | | | | | | | |
| RCF = 500 g/2 | 6 | stable | 27.07% | 500 | 2 | 0.248 | | 0.2123 |
| min | | | | | | | | |
| RCF = 500 g/4 | 7 | stable | 22.67% | 500 | 4 | 0.176 | | 0.1403 |
| min | | | | | | | | |
| RCF = 500 g/8 | 8 | stable | 24.26% | 500 | 8 | 0.158 | | 0.1223 |
| min | | | | | | | | |
| RCF = 200 g/1 | 9 | almost stable | 30.24% | 200 | 1 | 0.797 | | 0.7613 |
| min | | | | | | | | |
| RCF = 200 g/2 | 10 | almost stable | 30.39% | 200 | 2 | 0.371 | | 0.3353 |
| min | | | | | | | | |
| RCF = 200 g/4 | 11 | almost stable | 29.46% | 200 | 4 | 0.296 | | 0.2603 |
| min | | | | | | | | |
| RCF = 200 g/8 | 12 | almost stable | 30.06% | 200 | 8 | 0.211 | | 0.1753 |
| min | | | | | | | | |
| RCF = 3000 g/1 | 13 | very stable | 18.36% | 3000 | 1 | 0.116 | | 0.0803 |
| min | | | | | | | | |
| RCF = 3000 g/2 | 14 | very stable | 21.03% | 3000 | 2 | 0.089 | | 0.0533 |
| min | | | | | | | | |
| RCF = 3000 g/4 | 15 | very stable | 16.17% | 3000 | 4 | 0.083 | | 0.0473 |
| min | | | | | | | | |
| RCF = 3000 g/8 | 16 | very stable | 15.34% | 3000 | 8 | 0.0693 | | 0.0336 |
| min | | | | | | | | |
| RCF = 17000 g/40 | 17 | very stable | 9.01% | 17000 | 40 | 0.0422 | | 0.0065 |
| min | | | | | | | | |
| 3 kDa | 18 | | | | | 0.0078 | | (−0.0279) |
| centrifugal UF | | | | | | | | |
| permeate from | | | | | | | | |
| Feed #2 | | | | | | | | |
| MF of #17 | 19 | | | | | 0.0357 | | 0 |
| 0.22 um Nylon filter | | | | | | | | |
| (base-line for | | | | | | | | |
| Abs_600) | | | | | | | | |
| Feed #2 as is | 20 | | | | | 3.66 | | (3.6243) |
| (broth pH | | | | | | | | |
| 2.95/3x/70° C.) | | | | | | | | |

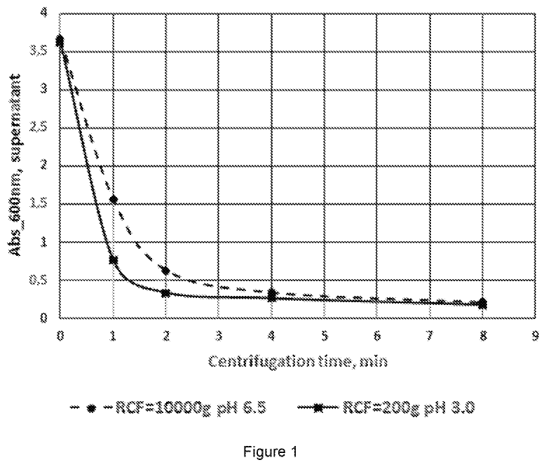

FIG. 1 illustrates a graph of centrifugation time compared to supernatant clarity according to OD600. Specifically, the figure shows clarification example vs centrifugation time at pH=3.0 (Feed #2) at RCF=200 g vs pH=6.5 (Feed #1) at RCF=10000 g. As shown, 50× lower RCF=200 g provides even better or similar OD600 at pH=3.0 vs high RCF=10000 g at pH=6.5.

### Example 6

Centrifugation at 1.4 kg Scale at pH=6.5 and 3.0

The remaining Feed #1 (pH=6.5/3×/70° C.) from example 4 and 5 in two 700 g equal portions was accelerated in Beckman Coulter Avanti J-26S XP centrifuge to RCF=10000 g (6330 RPM) in 2.5 min and exposed to the final 10000 g for 10 min. The obtained dark-brown non-transparent supernatant (m=1156 g) was removed by decantation. The obtained pellet was not stable and settled down horizontally from the walls. Feed #2 (pH=3.0/3×/70° C.) was processed in the same manner, but with acceleration to lower RCF=3000 g (3467 RPM) in 1.5 min and holding at RCF=3000 g for a shorter time of 2.5 min. The obtained clear pale brown-orange supernatant (m=1187 g) was separated to give stable pellet that was not settling down but remained stick tight to the walls. Samples of supernatants and pellets were analysed.

| | BWM | BWM | BWM | RCF, | time, | Abs_600 nm | |
|---|---|---|---|---|---|---|---|
| Description | #1 | #2 | average | g | min | Supernatant | OD600 |
| Supernatant Feed #1 | 17.34% | 17.38% | 17.36% | 10000 | 10 | 1.658 | 1.286 |
| @10000 g/10 min (pH 6.5) | | | | | | | |
| MF of the above supernatant, | | | | | | 0.372 | |
| filter RC 0.45 um | | | | | | | |
| Supernatant Feed #2 | 14.22% | 16.07% | 15.14% | 3000 | 2.5 | 0.069 | 0.0243 |
| @3000 g/3 min (pH 3.0) | | | | | | | |
| MF of the above supernatant, | | | | | | 0.0447 | |
| filter RC 0.45 um | | | | | | | |

### Example 7

### Settling Velocities vs pH, Dilution and Pre-Heat

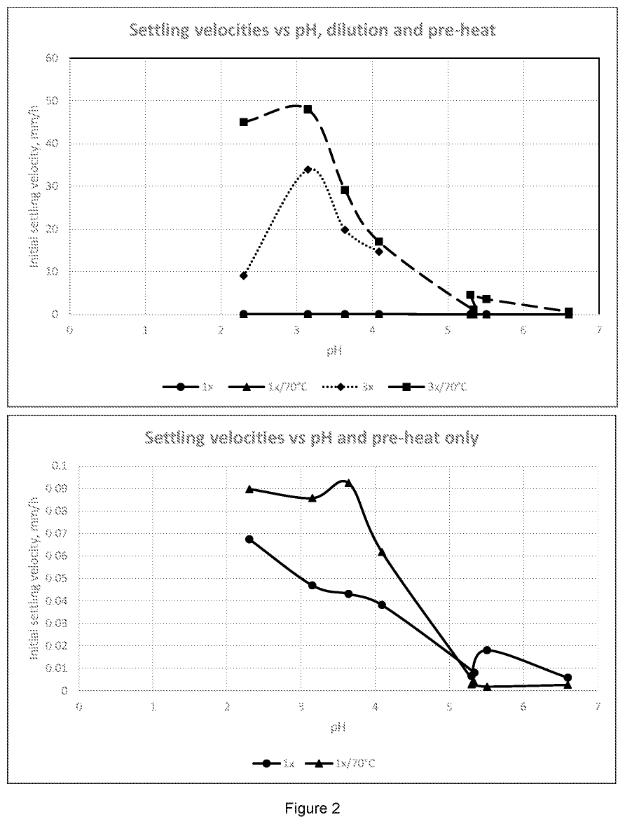

Procedure: LNFP-I-containing broth was generated as described in example 2, but without pH adjustment (pH=6.6). 100 ml (ca. 103 g) broth samples were pH-adjusted with 25% H₂SO₄ solution to give broth samples with 8 varied pH values from pH=2.3 to 6.6 as indicated in the table below. From each sample 4 further samples of 15 ml volume were prepared: one non modified, one 3× diluted (as described in example 3), one heat treated (30 min at 70° C. as described in example 3), and one 3× diluted and heat treated, thus resulting in 32 samples altogether. The obtained samples were placed vertically in NerbePlus centrifuge PP falcon tubes at ambient temperature (T=22)° C. The height of clear supernatant layer was periodically measured in mm vs time. The initial settling velocities were obtained from linear regression of height vs time using several initial points and summarized in the table below and in FIG. 2.

Settling velocities vs pH (dilution and pre-heat)

| | | | | final | | | | | initial | |
|---|---|---|---|---|---|---|---|---|---|---|
| | | 25% | | pH | | | | preheat | settling | |
| | broth | H₂SO₄ | | (next | Sample | H₂O | | 30 | velocity | |
| Sample | (g) | (ml) | pH | day) | (g) | (g) | dilution | min | (mm/h) | R² |
| #1 | 103.56 | 4.8 | 2.3 | 2.3 | 16.261 | 0 | No | No | 0.0674 | 0.9987 |
| #2 | 102.9 | 3.6 | 2.8 | 3.15 | 16.147 | 0 | No | No | 0.0469 | 0.9921 |
| #3 | 103.00 | 3.1 | 3.28 | 3.64 | 15.902 | 0 | No | No | 0.0431 | 0.9888 |
| #4 | 103.00 | 2.8 | 3.8 | 4.09 | 15.927 | 0 | No | No | 0.0382 | 0.9963 |
| #5 | 103.00 | 1.8 | 4.3 | 5.34 | 15.976 | 0 | No | No | 0.0081 | 0.994 |
| #6 | 103.03 | 1.8 | 5.2 | 5.3 | 15.949 | 0 | No | No | 0.0066 | 0.9673 |
| #7 | 103.12 | 1.4 | 5.3 | 5.51 | 16.074 | 0 | No | No | 0.0181 | 0.9793 |
| #8 | | 0 | 6.6 | 6.6 | 16.182 | 0 | No | No | 0.0059 | 0.9796 |
| #9 | | Sample #1 | | 2.3 | 16.294 | 0 | No | 70° C. | 0.0897 | 0.9985 |
| #10 | | Sample #2 | | 3.15 | 16.149 | 0 | No | 70° C. | 0.0857 | 0.9912 |
| #11 | | Sample #3 | | 3.64 | 15.91 | 0 | No | 70° C. | 0.0924 | 0.9872 |
| #12 | | Sample #4 | | 4.09 | 15.884 | 0 | No | 70° C. | 0.0617 | 0.9955 |
| #13 | | Sample #5 | | 5.34 | 15.986 | 0 | No | 70° C. | 0.0035 | 0.9478 |
| #14 | | Sample #6 | | 5.3 | 15.935 | 0 | No | 70° C. | 0.0029 | 0.7664 |
| #15 | | Sample #7 | | 5.51 | 15.913 | 0 | No | 70° C. | 0.0019 | 0.9685 |
| #16 | | Sample #8 | | 6.6 | 15.791 | 0 | No | 70° C. | 0.0027 | 0.96 |
| #17 | | Sample #1 | | 2.3 | 5.201 | 10.413 | 3x | No | 9.0439 | 0.9739 |
| #18 | | Sample #2 | | 3.15 | 5.183 | 10.371 | 3x | No | 33.90 | 0.9862 |
| #19 | | Sample #3 | | 3.64 | 5.096 | 10.234 | 3x | No | 19.796 | 0.9963 |
| #20 | | Sample #4 | | 4.09 | 5.003 | 10.064 | 3x | No | 14.674 | 0.9822 |
| #21 | | Sample #5 | | 5.34 | 5.011 | 10.04 | 3x | No | N/A: unclear | |
| #22 | | Sample #6 | | 5.3 | 5.024 | 10.112 | 3x | No | supernatant | |
| #23 | | Sample #7 | | 5.51 | 5.113 | 10.228 | 3x | No | | |
| #24 | | Sample #8 | | 6.6 | 5.178 | 10.374 | 3x | No | | |
| #25 | | Sample #1 | | 2.3 | 5.231 | 10.465 | 3x | 70° C. | 45 | 0.9643 |
| #26 | | Sample #2 | | 3.15 | 5.256 | 10.511 | 3x | 70° C. | 48 | 0.9552 |
| #27 | | Sample #3 | | 3.64 | 5.096 | 10.187 | 3x | 70° C. | 29.1 | 0.985 |
| #28 | | Sample #4 | | 4.09 | 5.009 | 10.05 | 3x | 70° C. | 17.014 | 0.9989 |
| #29 | | Sample #5 | | 5.34 | 5.005 | 10.116 | 3x | 70° C. | 1.08442 | 0.9414 |
| #30 | | Sample #6 | | 5.3 | 5.014 | 10.15 | 3x | 70° C. | 4.5759 | 0.9751 |
| #31 | | Sample #7 | | 5.51 | 5.025 | 10.095 | 3x | 70° C. | 3.5829 | 0.6434 |
| #32 | | Sample #8 | | 6.6 | 5.056 | 10.172 | 3x | 70° C. | 0.6377 | 1 |

The results show significant acceleration of settling velocities, i.e. to ca. 50 times after pH-adjustment to optimal pH around 3-3.5 vs initial pH between 6 and 7. In addition, dilution has a substantial effect as well with up to 500 times acceleration. Finally, simultaneous pH-adjustment and dilution have synergistic affect with up to 25000 times acceleration of settling velocities, i.e. to 30-50 mm/h vs 0.002-0.02 mm/h (without dilution and pH-adjustment). Pre-heating provides additional 1.4-2× acceleration and better clarification.

### Example 8

Continuous Industrial-Scale Centrifugation with Sedicanter S3

2′-FL-containing broth was generated by fermentation using a genetically modified E. coli strain of LacZ⁻, LacY⁺ phenotype, wherein said strain comprises a recombinant gene encoding an α-1,2-fucosyltransferase enzyme which is able to transfer fucose of GDP-fucose to the internalized lactose and genes encoding a biosynthetic pathway to GDP-fucose. The fermentation was performed by culturing the strain in the presence of exogenously added lactose and a suitable carbon source, thereby producing 2′-FL which was accompanied with DFL and unreacted lactose as major carbohydrate impurities in the fermentation broth.

Part of the obtained broth (7 m³) was acidified with H₂SO₄ to pH=3.6 and diluted with de-ionized water in 1:1 volume ratio (7 m³) and heated to 65° C. A first part of the pre-treated broth was then passed continuously at a 800-900 kg/h flow rate through Flottweg Sedicanter S3E at RCF=5000 g for 12.5 h. The next day, the centrifugation was continued with the remaining part of the broth at a 900 kg/h flow rate first at RCF=6500 g for 3 h then at 10000 g for additional 2.5 h. Samples were taken from the obtained clarified solution (centrate) and sediment at given time points and analysed for residual suspended solids by OD650, carbohydrate content by HPLC and protein content by Bradford test.

Continuous centrifugation with Sedicanter S3E: conditions and outcome

| feed | dilution (broth + water) | 1:1 (7 m³ + 7 m³) | | |
|---|---|---|---|---|
| | OD650 (broth as is: 115) | 65 | | |
| | temp (° C.) holding vessel | 65-66 | | |
| | pH | 3.6 | | |
| Sedicanter | RCF | 5000 g | 6500 g | 10000 g |
| parameters | feed rate (kg/h) | 800-900 | 900 | 900 |
| | torque (%) | 11.4 | 12.5 | 18.2-19.4 |
| | diff. speed (rpm) | 8.4 | 8.2 | 8.0 |
| | centrate pressure (bar) | | 1.6 | |
| sediment | total dry weight (%, avg.) | 34 | 31.5 | 40 |
| centrate | OD650 (avg.) | 0.8 | 0.35 | 0.035 |
| | biomass removal (% by OD) | 98.8 | 99.5 | 99.9 |

| | HPLC, g/kg for centrates or | | | flow | | 2′-FL |
|---|---|---|---|---|---|---|
| sample | wt % for sediment samples | | | rate | 2′-FL | yield in |
| description | lactose | 2′-FL | DFL | (kg/h) | (kg/h) | centrate |
| feed for | 1.63 | 47.37 | 1.20 | 800 | 37.9 | |
| Sedicanter | | | | | | |
| centrate/5000 g | 1.77 | 50.93 | 1.26 | 700 | 35.6 | 92.2% |
| after 8 h | | | | | | |
| sediment/5000 g | 0.10% | 3.00% | 0.07% | 100 | 3.0 | |
| after 8 h | | | | | | |
| centrate/5000 g | 1.78 | 51.60 | 1.23 | 790 | 40.8 | 91.2% |
| after 12 h | | | | | | |
| sediment/5000 g | 0.12% | 3.59% | 0.08% | 110 | 3.9 | |
| after 12 h | | | | | | |
| feed for | 1.80 | 51.84 | 1.27 | 900 | 46.7 | |
| Sedicanter | | | | | | |
| centrate/6500 g | 1.79 | 51.22 | 1.23 | 800 | 41.0 | 91.8% |
| after 1 h | | | | | | |
| sediment/6500 g | 0.11% | 3.65% | 0.08% | 100 | 3.7 | |
| after 1 h | | | | | | |
| centrate/6500 g | 1.85 | 50.17 | 1.22 | 800 | 40.1 | 91.5% |
| after 3 h | | | | | | |
| sediment/6500 g | 0.12% | 3.73% | 0.08% | 100 | 3.7 | |
| after 3 h | | | | | | |
| centrate/10000 g | 1.88 | 49.01 | 1.23 | 800 | 39.2 | 91.9% |
| after 4 h | | | | | | |
| sediment/10000 g | 0.12% | 3.44% | 0.08% | 100 | 3.4 | |
| after 4 h | | | | | | |
| centrate/10000 g | 1.87 | 50.60 | 1.24 | 800 | 40.5 | 91.8% |
| after 5 h | | | | | | |
| sediment/10000 g | 0.14% | 3.60% | 0.09% | 100 | 3.6 | |
| after 5 h | | | | | | |

The obtained sediment (1.2 m³) was re-suspended with water (3.9 m³) and re-processed again in Sedicanter at 65° C.(centrifugation time: 6 h).

Continuous centrifugation with Sedicanter S3E, re-suspension: conditions and outcome

| feed | dilution (sediment + water) | | | 1:3 (1.2 m³ + 3.9 m³) | | |
|---|---|---|---|---|---|---|
| | OD650 (broth as is: 115) | | | 140 | | |
| | temp (° C.) holding vessel | | | 65-66 | | |
| | pH | | | 3.65 | | |
| Sedicanter | RCF | | | 5000 g | | |
| parameters | feed rate (kg/h) | | | 1100 | | |
| | torque (%) | | | 16.6 | | |
| | diff. speed (rpm) | | | 17.3 | | |
| | centrate pressure (bar) | | | 1.5 | | |
| sediment | total dry weight (%, avg.) | | | 24.4 | | |
| centrate | OD650 (avg.) | | | 0.06 | | |
| | biomass removal (% by OD) | | | 99.9 | | |
| | HPLC, g/kg for centrates or wt % | | | flow | | 2′-FL |
| sample | for sediment samples | | | rate | 2′-FL | yield in |
| description | lactose | 2′-FL | DFL | (kg/h) | (kg/h) | centrate |
| feed for Sedicanter | 0.40 | 12.04 | 0.29 | 1100 | 13.2 | |
| centrate/5000 g | 0.47 | 12.92 | 0.30 | 870 | 11.2 | 84.8% |
| after 1 h | | | | | | |
| sediment/5000 g | 0.04% | 0.88% | 0.02% | 230 | 2.0 | |
| after 1 h | | | | | | |
| centrate/5000 g | 0.46 | 12.74 | 0.29 | 870 | 11.1 | 83.6% |
| after 4 h | | | | | | |
| sediment/5000 g | 0.04% | 0.95% | 0.02% | 230 | 2.2 | |
| after 4 h | | | | | | |

Another part of the fermentation broth was diluted in 1:2 ratio (2 m³ broth+4 m³ water) and processed at 65° C. at RCF=5000 g for 4 h then at RCF=6500 g for 3 h.

Continuous centrifugation with Sedicanter S3E: conditions and outcome

| feed | dilution (broth + water) | 1:2 (2 m³ + 4 m³) | |
|---|---|---|---|
| | OD650 (broth as is: 115) | 48 | |
| | temp (° C.) holding vessel | 65-66 | |
| | pH | 3.83 | |
| Sedicanter | RCF | 5000 g | 6500 g |
| parameters | feed rate (kg/h) | 1000 | 1000 |
| | torque (%) | 14-15.3 | 17.4 |
| | diff. speed (rpm) | 17.4 | 17.3 |
| | centrate pressure (bar) | 1.1-1.4 | 1.6 |
| sediment | total dry weight (%, avg.) | 30 | 30 |
| centrate | OD650 (avg.) | 0.6 | 0.135 |
| | biomass removal (% by OD) | 98.75 | 99.7 |

| | HPLC, g/kg for centrates or wt % | | | flow | | 2′-FL |
|---|---|---|---|---|---|---|
| sample | for sediment samples | | | rate | 2′-FL | yield in |
| description | lactose | 2′-FL | DFL | (kg/h) | (kg/h) | centrate |
| feed for | 1.07 | 33.37 | 0.80 | 1000 | 33.4 | |
| Sedicanter | | | | | | |
| centrate/5000 g | 1.20 | 35.99 | 0.91 | 890 | 32.0 | 92.4% |
| after 2 h | | | | | | |
| sediment/5000 g | 0.09% | 2.41% | 0.06% | 110 | 2.6 | |
| after 2 h | | | | | | |
| centrate/5000 g | 1.28 | 35.97 | 0.91 | 900 | 32.4 | 93.0% |
| after 2.5 h | | | | | | |
| sediment/5000 g | 0.09% | 2.44% | 0.06% | 100 | 2.4 | |
| after 2.5 h | | | | | | |
| centrate/6500 g | 1.28 | 35.21 | 0.87 | 900 | 31.7 | 93.0% |
| after 5 h | | | | | | |
| sediment/6500 g | 0.09% | 2.39% | 0.06% | 100 | 2.4 | |
| after 5 h | | | | | | |
| centrate/6500 g | 1.20 | 36.59 | 0.89 | 900 | 32.9 | 93.2% |
| after 6 h | | | | | | |
| sediment/6500 g | 0.09% | 2.39% | 0.06% | 100 | 2.4 | |
| after 6 h | | | | | | |

In another run, the fermentation broth with a 1:2 dilution (3 m³ broth+6 m³ water) was further acidified to pH=3.3 with sulfuric acid and processed at 65° C. at RCF=5000 g for 4 h then at RCF=6500 g for 6 h.

Continuous centrifugation with Sedicanter S3E: conditions and outcome

| feed | dilution (broth + water) | 1:2 (3 m³ + 6 m³) | |
|---|---|---|---|
| | OD650 (broth as is: 115) | 50 | |
| | temp (° C.) holding vessel | 65-66 | |
| | pH | 3.32 | |
| Sedicanter | RCF | 5000 g | 6500 g |
| parameters | feed rate (kg/h) | 1000 | 1000 |
| | torque (%) | 16 | 18.2 |
| | diff. speed (rpm) | 17.3 | 17.3 |
| | centrate pressure (bar) | 1.55 | 1.55 |
| sediment | total dry weight (%, avg.) | 30 | 31 |
| centrate | OD650 (avg.) | 0.15 | 0.075 |
| | biomass removal (% by OD) | 99.7 | 99.85 |

| | HPLC, g/kg for centrates or | | | flow | | 2′-FL |
|---|---|---|---|---|---|---|
| sample | wt % for sediment samples | | | rate | 2′-FL | yield in |
| description | lactose | 2′-FL | DFL | (kg/h) | (kg/h) | centrate |
| feed for | 1.35 | 39.79 | 0.97 | 1000 | 39.8 | |
| Sedicanter | | | | | | |
| centrate/5000 g | 1.30 | 36.60 | 0.92 | 900 | 32.9 | 93.3% |
| after 2.5 h | | | | | | |
| sediment/5000 g | 0.09% | 2.38% | 0.06% | 100 | 2.4 | |
| after 2.5 h | | | | | | |
| centrate/5000 g | 1.36 | 37.53 | 0.93 | 900 | 33.8 | 94.5% |
| after 4 h | | | | | | |
| sediment/5000 g | 0.08% | 1.96% | 0.05% | 100 | 2.0 | |
| after 4 h | | | | | | |
| centrate/6500 g | 1.40 | 37.60 | 0.93 | 900 | 33.8 | 93.2% |
| after 7.5 h | | | | | | |
| sediment/6500 g | 0.10% | 2.46% | 0.06% | 100 | 2.5 | |
| after 7.5 h | | | | | | |
| centrate/6500 g | 1.39 | 36.20 | 0.89 | 900 | 32.6 | 93.1% |
| after 10 h | | | | | | |
| sediment/6500 g | 0.10% | 2.41% | 0.07% | 100 | 2.4 | |
| after 10 h | | | | | | |

In all cases, the suspended solid content in the clarified centrates was reduced by 99% or above. The recovery yield of 2′-FL was 91-95% after single centrifugation step and around 99% after re-suspension (cumulative). The tests clearly demonstrate efficient biomass removal and clarification of bacterial fermentation broth by continuous centrifugation at industrial scale and at relatively low g-force (5000 g) after optimized broth pre-treatment conditions as disclosed in previous lab-scale examples 1-7.

### Example 9

### UF Performance After Centrifugation

This example demonstrates that biomass removal throughput could be substantially improved by 4-7 times if centrifugation was performed before ultrafiltration (UF) compared to UF alone.

The centrate generated in the previous example with the largest amount of residual suspended solids was selected for further UF clarification trials (1:1 dilution, RCF=5000 g). A portion of the centrate was processed under different conditions in the MMS SW18 membrane filtration system equipped with Kerasep BE membrane of 15 kDa cut-off, area 0.21 m² at 0.5 m/s cross-flow velocity (400 l/h cross-flow) with varying trans membrane pressure (TMP) and temperature. In each test, the centrate of ca. 5 kg was equilibrated for 20-30 min under chosen conditions, then the permeate was collected until a retentate mass reduction by a factor of 5 (concentration factor (CF)=5). Permeate fluxes were recorded and average fluxes are summarized in the table below.

UF fluxes after centrifugation

| | | | average flux (LMH) vs temperature and TMP, CF 1→5 | | | | | |
|---|---|---|---|---|---|---|---|---|
| | | protein | T = 15° C., | T = 15° C., | T = 15° C., | T = 60° C., | T = 60° C., | T = 60° C., |
| feed | OD600 | (mg/l) | TMP = 0.6 | TMP = 2.9 | TMP = 6.0 | TMP = 0.9 | TMP = 2.9 | TMP = 6.0 |
| centrate | 0.62 | 93.8 | 19.9 | 66.1 | 102.5 | 46.9 | 117.7 | 183.7 |

Then a larger portion of centrate (27.1 kg) was equilibrated at TMP=3.0 bar, cross-flow 0.9 m/s (600 l/h) and T=60° C. for 3 h with permeate re-circulation back to the feed tank. The permeate flux was measured periodically with initial flux of 250 l/m²h (LMH) at time=0 which was reduced and stabilized at 120 LMH and time=3 h. Then 25.75 kg permeate was collected in 100 min (CF=20) with the flux drop further to 40 LMH in the end with the calculated average flux of 74 LMH.

For comparison, initial 2′-FL broth before centrifugation was processed several times at 60° C., cross-flow 1-5 m/s, TMP 1-3 bar and CF-2 with continuous diafiltration with 1-2 volumes of deionized water to give average permeate flux values of 10-20 LMH, which are significantly lower (4-7 times) than the permeate fluxes observed after centrifugation.

### GENERAL

The following figures and examples are provided below to illustrate the present disclosure. They are intended to be illustrative and are not to be construed as limiting in any way.

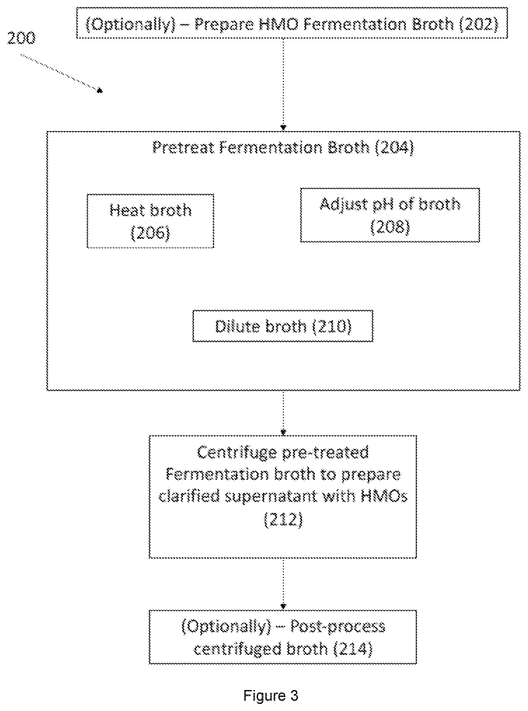

FIG. 3 illustrates an embodiment of a method (200) for isolation/purifying a fermentation for a HMOs. As shown, the original broth having the HMOs can be prepared (202). This may be performed as part of the method, or the broth can be provided from another source into the method.

Once the broth is obtained, the broth can then be pre-treated (204). This can be performed as discussed in detail above. For example, the broth can be heated (206), have its pH adjusted (208), and or diluted (210). One, two, or all of the pre-treatment steps can be performed. Following the pre-treatment, the broth can then be centrifuged (212). This can be performed on any number of centrifuging processes, and the method is not limited to any particular type of centrifuging process. The centrifuging can separate the HMOs away from any other components, such as a biomass. Once centrifuging is done, the HMOs can have been isolated and/or purified from the broth.

Optionally, further post-processing (214) can be performed to further obtain the HMOs. These post-processing techniques have been detailed above. In alternative embodiments, no post-processing is performed, and the method can end after the centrifuging. For example, the HMOs may be collected from the centrifuged broth.

### EXAMPLE EMBODIMENTS

Discussed below are certain non-limiting example embodiments of one or more exemplary methods as disclosed herein.

Embodiment 1. A method for purification of human milk oligosaccharides (HMOs) from a suspension containing one or more of biomass and proteins, the method comprising:
pre-treating the suspension via pH-adjustment, dilution and/or heat treatment, preferably pH-adjustment, dilution and heat treatment; and
centrifuging the suspension after the pre-treatment,
   thereby forming a clarified supernatant containing the purified HMOs.

Embodiment 2. The method according to Embodiment 1, wherein the pH-adjustment or acidification of the suspension is to a pH between 2 and 5, preferably between 3 and 4.

Embodiment 3. The method according to any one of the preceding Embodiments, wherein the dilution is performed so that
the mass of the diluted suspension is between 1.1 and 10 times of the original mass of the suspension, preferably between 1.5 and 10, more preferably between 2 and 4, more preferably between 2.5 and 3.5, or
the volume of the diluted suspension is between 2 and 10 times of the original volume of the suspension, preferably between 2 and 8, more preferably between 2 and 4, more preferably between 2.5 and 3.5,

Embodiment 4. The method according to Embodiment 3, wherein the BWM of the diluted suspension is less than 20, 15, 10 or 5%, preferably around 10-15%.

Embodiment 5. The method according to any one of the preceding Embodiments, wherein the suspension is heated to 45-120° C., preferably 60-90° C., more preferably 60-80° C.

Embodiment 6. The method according to any one of the preceding Embodiments, wherein the centrifuging is a continuous process with a continuous feed addition, and wherein the centrifuging further comprises a continuous or periodical wet solid removal.

Embodiment 7. The method according to any one of the preceding Embodiments, wherein the centrifuging is performed at RCF<10000 g for a residence time of <3 minutes.

Embodiment 8. The method according to any one of the preceding Embodiments, wherein the centrifuging is performed at RCF<20000 g for a residence time of <3 minutes.

Embodiment 9. The method according to any one of the preceding Embodiments, wherein the centrifuging is performed with a working volume of >10 l and a feed flow rate of >1000 l/h for a residence time of 3 minutes or less.

Embodiment 10. The method according to any one of the preceding Embodiments, wherein the centrifuging is performed with a working volume of >10 l and a feed flow rate of >1000 l/h for a residence time of 6 minutes or less.

Embodiment 11. The method according to any one of the preceding Embodiments, wherein the centrifuging is by a Sedicanter, preferably with a RCF of 5000 to 10000 g.

Embodiment 12. The method according to any one of the preceding Embodiments, wherein centrifuging is by a conical plate centrifuge.

Embodiment 13. The method according to any one of the preceding Embodiments, wherein centrifuging is by a solid bowl (decanter) centrifuge.

Embodiment 14. The method according to any one of the preceding Embodiments, wherein the clarified supernatant comprises OD600<0.1, residual proteins <500 mg/l, and a product yield >80% after a single instance of the centrifuging.

Embodiment 15. The method according to any one of the preceding Embodiments, wherein the clarified supernatant is further purified by at least one of the following steps:
a. Ultrafiltration or microfiltration of the clarified supernatant with membrane having a pore size <1.0 μm and MWCO >600 Da and an optional diafiltration, wherein the HMOs are collected in a permeate stream;
b. Nanofiltration with a membrane having a MWCO 100-3500 Da and an optional diafiltration, wherein a majority of the HMOs are retained in retentate thereby allowing at least water and preferably other small molecules to pass through the membrane, the small molecules including monosaccharides, disaccharides, small bacterial metabolites, and salts;
c. Adsorption of the HMOs from the clarified supernatant on active charcoal where an amount of active charcoal relative to the HMOs is >100% by weight, followed by elution of a purified HMO product stream with aqueous organic solvent selected from alcohol C1-C4, acetic acid, or acetonitrile;
d. Polishing de-coloration of the clarified supernatant by active charcoal, wherein an amount of charcoal relative to the HMOs is <100% by weight, preferably <10% allowing most of the HMOs to pass while retaining residual biomolecules, coloured compounds, and other hydrophobic molecules;
e. Concentration by evaporation, vacuum evaporation, nanofiltration, reverse osmosis or forward osmosis;
f. Solid HMO separation via crystallization;
g. Solid HMO separation via water removal by freeze-drying, spray-drying, drum drying, or belt drying the HMOs;
h. Sterile filtration of the HMOs;
i. Electrodialysis of the HMOs;
j. Performing cationic and/or anionic exchange of the HMOs;
k. Performing de-salting by passing through a cationic exchanger in H⁺-form followed by acid adsorbing step by passing through or adding weakly basic resin in a free base form;
l. Performing de-salting by dialysis or neutralization dialysis;
m. Performing Chromatography separation of the HMOs; and

Embodiment 16. The method according to any one of the preceding Embodiments, wherein the suspension is produced by genetically modified E. coli capable of producing the HMOs.

Embodiment 17. The method according to any one of the preceding Embodiments, with the proviso that a flocculation agent is not used

Embodiment 18. The method according to any one of the preceding Embodiments, wherein the centrifugation is performed at an industrial scale.

Embodiment 19. The method according to any one of the preceding Embodiments, wherein the pre-treating the suspension is via pH-adjustment, dilution, and heat treatment.

Embodiment 20. The method according to any one of the preceding Embodiments, wherein the pre-treating the suspension is via pH-adjustment and dilution. Embodiment 21. The method according to any one of the preceding Embodiments, wherein the pre-treating the suspension is via pH-adjustment followed by dilution. Embodiment 22. The method according to any one of the preceding Embodiments, wherein the pre-treating the suspension is via dilution followed by pH-adjustment.

Embodiment 23. The method according to any one of the preceding Embodiments, wherein the pre-treating the suspension is via pH-adjustment and heat treatment.

Embodiment 24. The method according to any one of the preceding Embodiments, wherein the pre-treating the suspension is via pH-adjustment followed by heat treatment.

Embodiment 25. The method according to any one of the preceding Embodiments, wherein the pre-treating the suspension is via heat treatment followed by pH-adjustment.

Embodiment 26. The method according to any one of the preceding Embodiments, wherein the pre-treating the suspension is via dilution and heat treatment.

Embodiment 27. The method according to any one of the preceding Embodiments, wherein the pre-treating the suspension is via dilution followed by heat treatment.

Embodiment 28. The method according to any one of the preceding Embodiments, wherein the pre-treating the suspension is via heat treatment followed by dilution.

Embodiment 29. The method according to any one of the preceding Embodiments, wherein the pre-treating the suspension is via concurrent pH-adjustment and heat treatment.

Embodiment 30. The method according to any one of the preceding Embodiments, wherein the pre-treating the suspension is via concurrent pH-adjustment and heat treatment followed by dilution.

Embodiment 31. The method according to any one of the preceding Embodiments, wherein the pre-treating the suspension is via dilution followed by concurrent pH-adjustment and heat treatment.

Embodiment 32. The method according to any one of the preceding Embodiments, wherein the pre-treating the suspension is via concurrent pH-adjustment and dilution.

Embodiment 33. The method according to any one of the preceding Embodiments, wherein the pre-treating the suspension is via concurrent pH-adjustment and dilution followed by heat treating.

Embodiment 34. The method according to any one of the preceding Embodiments, wherein the pre-treating the suspension is via heat treating followed by concurrent pH-adjustment and dilution.

Embodiment 35. The method according to any one of the preceding Embodiments, wherein the pre-treating the suspension is via concurrent dilution and heat treatment.

Embodiment 36. The method according to any one of the preceding Embodiments, wherein the pre-treating the suspension is via concurrent dilution and heat treatment followed by pH-adjustment.

Embodiment 37. The method according to any one of the preceding Embodiments, wherein the pre-treating the suspension is via pH-adjustment followed by concurrent dilution and heat treatment.

Embodiment 38. The method according to any one of the preceding Embodiments, wherein the pre-treating the suspension is via pH-adjustment followed by dilution followed by heat treatment.

Embodiment 39. The method according to any one of the preceding Embodiments, wherein the pre-treating the suspension is via pH-adjustment followed by heat treatment followed by heat dilution.

Embodiment 40. The method according to any one of the preceding Embodiments, wherein the pre-treating the suspension is via dilution followed by pH -adjustment followed by heat treatment.

Embodiment 41. The method according to any one of the preceding Embodiments, wherein the pre-treating the suspension is via dilution followed by heat treatment followed by pH -adjustment.

Embodiment 42. The method according to any one of the preceding Embodiments, wherein the pre-treating the suspension is via heat treatment followed by dilution followed by pH -adjustment.

Embodiment 43. The method according to any one of the preceding Embodiments, wherein the pre-treating the suspension is via heat treatment followed by pH-adjustment followed by dilution.

Embodiment 44. The method according to any one of the preceding Embodiments, wherein the HMO is a neutral HMO.

Embodiment 45. The method according to Embodiment 44, wherein the neutral HMO is selected from the group consisting of 2′-FL, 3-FL, DFL, LNT, LNnT, LNFP-I, LNFP-II, LNFP-III, LNFP-V and LNFP-VI.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the exemplary embodiments may be implemented at least in part by means of both hardware and software, and that several “means”, “units” or “devices” may be represented by the same item of hardware.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

1. A method for purification of a human milk oligosaccharide (HMO) from a suspension containing one or more of a biomass and proteins, the method comprising:
pre-treating the suspension via pH-adjustment, dilution and/or heat treatment; and
centrifuging the suspension after the pre-treatment, thereby forming a clarified supernatant containing the purified HMO.

2. The method according to claim 1, wherein the suspension is a fermentation broth.

3. The method according to claim 1, wherein the pre-treatment comprises pH-adjustment, dilution and heat treatment.

4. The method according to claim 1, wherein the pH-adjustment is acidification of the suspension to a pH between 2 and 5, preferably between 3 and 4.

5. The method according to claim 1, wherein the dilution is performed so that the volume of the diluted suspension is between 2 and 10 times of the original volume of the suspension.

6. The method according to claim 5, wherein the bio-wet-mass (BWM) of the suspension after dilution is less than 20%.

7. The method according to claim 1, wherein the heat treatment comprises heating the suspension to 45-120° C.

8. The method according to claim 1, wherein the centrifuging is a continuous process with a continuous feed addition, and wherein the centrifuging further comprises a continuous or periodical wet solid removal.

9. The method according to claim 1, wherein the centrifuging is by a conical plate centrifuge .

10. The method according to claim 1, wherein the centrifuging is by a solid bowl (decanter) centrifuge.

11. The method according to claim 1, wherein the clarified supernatant obtained after centrifuging is further purified by ultrafiltration.

12. The method according to claim 1, wherein the clarified supernatant obtained after centrifuging is further purified by
a) optional ultrafiltration (UF)
b) nanofiltration (NF), and
c) treatment with an ion exchange resin, and/or chromatography on a neutral solid phase.

13. The method according to claim 1, wherein the HMO is a neutral HMO.

14. The method according to claim 13, wherein the neutral HMO is selected from the group consisting of 2′-FL, 3-FL, DFL, LNT, LNnT, LNFP-I, LNFP-II, LNFP-III, LNFP-V and LNFP-VI.

15. The method according to claim 13, wherein the neutral HMO is selected from the group consisting of 2′-FL, 3-FL, DFL and LNFP-I.

16. The method according to claim 13, wherein the neutral HMO is 2′-FL or LNFP-I.

17. The method according to claim 13, wherein the neutral HMO is LNT or LNnT.

18. The method according to claim 4, wherein the pH-adjustment is acidification of the suspension to a pH between 3 and 4.

19. The method according to claim 5, wherein the dilution is performed so that the volume of the diluted suspension is between 2 and 4 of the original volume of the suspension.

20. The method according to claim 7, wherein the heat treatment comprises heating the suspension to 60-90° C.
